(19) United States
(12) Patent Application Publication
(10) Pub. No.: US 2003/0194801 A1
(43) Pub. Date: Oct. 16, 2003

(54) Use of flavivirus for the expression of protein epitopes and development of new live attenuated vaccine virus to immune against flavivirus and other infectious agents

(76) Inventors: Mirna C. Bonaldo, Rio de Janeiro (BR); Ricardo Galler, Rio de Janeiro (BR); Marcos da Silva Freire, Rio de Janeiro (BR); Richard C. Garrat, Sao Paulo (BR)

Correspondence Address: NIXON & VANDERHYE, PC, 1100 N GLEBE ROAD, 8TH FLOOR, ARLINGTON, VA, 22201-4714, US

(21) Appl. No.: 10/275,707

(22) PCT Filed: Mar. 8, 2002
(86) PCT No.: PCT/BR02/00036

(30) Foreign Application Priority Data
Mar. 9, 2001 (GB) 0105877.5

(51) Int. Cl. C12Q 1/68; C12P 21/06; C12N 15/00; C12N 15/09; C12N 15/63; C12N 15/70; C12N 15/74; C12N 5/06; C12N 5/16

(52) U.S. Cl. 435/320.1; 435/6; 435/69.1; 435/345

(57) ABSTRACT

The present invention relates to a vaccine against infections caused by flavivirus. More particularly to the use of the YF vaccine virus (17D) to express at the level of its envelope, protein epitopes from other pathogens which will elicit a specific immune response to the parental pathogen.

The present invention relates to a vaccine against infections caused by flavivirus. More particularly to the use of the YF vaccine virus (17D) to express at the level of its envelope, protein epitopes from other pathogens which will elicit a specific immune response to the parental pathogen. There are provided new plasmids which were deposited on Dec. 21, 2000 under the following accesion number with the American Type Culture Collection (ATCC), 10801 University Blvd., Manassas, Va. 20110-2209: (i) pYFE200/13 accession number PTA-2854; (ii) pYF17D/8 accession number PTA-2855; (iii) pYFE200 accession number PTA-2856; (iv) pYFE200/8 accession number PTA-2857 and (v) pYFE200/1 accession number PTA-2858.

### BACKGROUND OF THE INVENTION

Flaviviruses consists of 70 serologically cross-reactive, closely related human or veterinary pathogens causing many serious illnesses, which includes dengue fever, Japanese encephalitis (JE), tick-borne encephalitis (TBE) and yellow fever (YF). The Flaviviruses are spherical viruses with 40-60 nm in diameter with an icosahedral capsid which contains a single positive-stranded RNA molecule.

YF virus is the prototype virus of the family of the Flaviviruses with a RNA genome of 10,862 nucleotides (nt), having a 5′ CAP structure and a short 5′ end nontranslated region (118 nt) and a nonpolyadenylated nontranslated 3′ end (511 nt). The first complete nucleotide sequence of a flavivirus genome was determined on the genome of the YF 17D-204 vaccine strain virus by Rice et al (Rice C. M.; Lenches, E.; Eddy, S. R.; Shin, S. J.; Sheets, R. L. and Strauss, J. H. 1985. “Nucleotide sequence of yellow fever virus: implications for flavivirus gene expression and evolution”. Science. 229: 726-733).

The single RNA is also the viral message and its translation in the infected cell results in the synthesis of a polyprotein precursor of 3,411 amino acids which is cleaved by proteolytic processing to generate 10 virus-specific polypeptides. From the 5′ terminus, the order of the encoded proteins is: C; prM/M; E; NS1; NS2A; NS2B; NS3; NS4A; NS4B and NS5. The first 3 proteins constitute the structural proteins, that is, form the virus together with the packaged RNA molecule and were named capsid (C, 12-14 kDa), membrane (M, and its precursor prM, 18-22 kDa) and envelope (E,52-54 kDa) all being encoded in the first quarter of the genome. The remainder of the genome codes for the nostructural proteins (NS) numbered in the order of synthesis from 1 through 5.

Three large nonstructural proteins have highly conserved sequences among flaviruses, NS1 (38-41 kDa), NS3 (68-70 kDa) and NS5 (100-103 kDa). A role in the replication of the negative strand RNA has been assigned to NS1 (Muylaert I R, Chambers T J, Galler R, Rice C M 1996. Mutagenesis of N-linked glycosylation sites of YF virus NS1: effects on RNA accumulation and mouse neurovirulence. Virology 222, 159-168; Muylaert I R; Galler R, Rice C M 1997. Genetic analysis of Yellow Fever virus NS1 protein: identification of a temperature-sensitive mutation which blocks RNA accumulation. J. Virol 71, 291-298; Lindenbach B D, Rice C M 1999. Genetic interaction of flavivirus nonstructural proteins NS1 and NS4A as a determinant of replicase function J. Virol. 73, 4611-4621; Lindenbach B D, Rice C M 1997. trans-complementation of yellow fever virus NS1 reveals a role in early RNA replication J. Virol. 71, 9608-9617).

NS3 has been shown to be bifunctional with a protease activity needed for the processing of the polyprotein at sites the cellular proteases will not (Chambers T J, Weir R C, Grakoui A, McCourt D W, Bazan J F, Fletterick R J, Rice C M 1990b. Evidence that the N-terminal domain of nonstructural protein NS3 from yellow fever virus is a serine protease responsible for site-specific cleavages in the viral polyprotein. Proc.Natl.Acad.Sci. USA 87, 8898-8902; Falgout B, Pethel M, Zhang Y M, Lai C J 1991. Both nonstructural proteins NS2B and NS3 are required for the proteolytic processing of dengue virus nonstructural proteins. J. Virol 65, 2467-2475; Yamshichikov V F, Compans R W 1995. Formation of the flavivirus envelope: role of the viral NS2B-NS3 protease. J. Virol. 69, 1995-2003; Yamshichikov V F; Trent, D W, Compans R W 1997. Upregulation of signalase processing and induction of prM-E secretion by the flavivirus NS2B-NS3 protease: roles of protease components. J. Virol. 71, 4364-4371; Stocks C E, Lobigs M 1998. Signal peptidase cleavage at the flavivirus C-prM junction: dependence on the viral NS2B-3 protease for efficient processing requires determinants in C, the signal peptide and prM. J. Virol. 72, 2141-2149). It also contains nucleotide triphosphatase/helicase activities (Gorbalenya A E, Koonin E V, Donchenko A P, Blinov V M 1989. Two related superfamilies of putative helicases involved in replication, recombination repair and expression of DNA and RNA genomes. Nucl.Acids.Res. 17, 4713-4730; Wengler and Wengler, 1993) being therefore also associated with viral RNA replication. NS5, the largest and most conserved viral protein, contains several sequence motifs believed to be common to viral RNA polymerases (Chambers T J, Hahn C S, Galler R, C M Rice 1990a. Flavivirus genome organization, expression and evolution. Ann.Rev.Microbiol. 44, 649-688; O'Reilly E K, Kao C C 1998. Analysis of RNA-dependent RNA polymerase structure and function as guided by known polymerase structures and computer predictions of secondary structure. Virology 252, 287-303) and exhibits RNA-dependent RNA polymerase activity (Steffens S, Thiel H J, Behrens S E 1999. The RNA-dependent RNA polymerases of different members of the family Flaviviridae exhibit similar properties in vitro. J. Gen. Virol. 80, 2583-2590). Finally, a number of cis and trans acting elements in flavivirus RNA replication have been identified (Kromykh A A, Sedlak P L, Westaway E G 2000. cis- and trans-acting elements in flavivirus RNA replication. J. Virol. 74, 3253-3263).

The 4 small proteins NS2A, NS2B, NS4A and NS4B are poorly conserved in their amino acid sequences but not in their pattern of multiple hydrophobic stretches. NS2A has been shown to be required for proper processing of NS1 (Falgout B, Channock R, Lai C J 1989. Proper processing of dengue virus nonstructural glycoprotein NS1 requires the N-terminal hydrophobic signal sequence and the downstream nonstructural protein NS2A. J. Virol. 63, 1852-1860) whereas NS2B has been shown to associate with NS3 to constitute the active viral protease complex (Chambers T J, Nestorowicz A, Amberg S M, Rice C M 1993. Mutagenesis of the yellow fever virus nonstructural polyportein: a catalitically active NS3 proteinase domain and NS2B are required for cleavages at dibasic sites. J. Virol. 65, 6797-6807; Falgout B, Pethel M, Zhang Y M, Lai C J 1991. Both nonstructural proteins NS2B and NS3 are required for the proteolytic processing of dengue virus nonstructural proteins. J. Virol 65, 2467-2475; Jan L R Yang C S, Trent D W, Falgout B, Lai C J 1995. Processing of Japanese encephalitis virus non-structural proteins:NS2B-NS3 complex and heterologous proteases. J. Gen. Virol. 76, 573-580). NS4A has been suggested to interact with NS1 integrating it into the cytoplasmic process of RNA replication (Lindenbach and Rice, 1999). Since viral RNA synthesis takes place in the cytosol in association with RER membranes it has been postulated that these hydrophobic proteins would be embedded in membranes and through protein-protein interactions participate in viral replication complexes together with NS3 and NS5 (Rice C M 1996. Flaviviridae: the viruses and their replication. In B N Fields, D M Knipe, P M Howley (eds), Fields Virology 3rd ed, Raven Press, USA, p. 931-960).

Two strains of yellow fever virus (YF), isolated in 1927, gave rise to the vaccines to be used for human immunization. One, the Asibi strain, was isolated from a young african named Asibi by passage in Rhesus monkey (Macaca mulatia), and the other, the French Viscerotropic Virus (FVV), from a patient in Senegal.

In 1935, the Asibi strain was adapted to growth in mouse embryonic tissue. After 17 passages, the virus, named 17D, was further cultivated until passage 58 in whole chicken embryonic tissue and thereafter, until passage 114, in denervated chicken embryonic tissue only. Theiler and Smith (Theiler M and Smith H H. 1937. The effect of prolonged cultivation in vitro upon the pathogenicity of yellow fever virus. J Exp Med. 65, 767-786) showed that, at this stage, there was a marked reduction in viral viscero and neurotropism when inoculated intracerebrally in monkeys. This virus was further subcultured until passages 227 and 229 and the resulting viruses, without human immune serum, were used to immunize 8 human volunteers with satisfactory results, as shown by the absence of adverse reactions and seroconversion to YF in 2 weeks (Theiler M, Smith H H 1937. The use of yellow fever virus modified by in vitro cultivation for human immunization J. Exp. Med 65:787-800).

In the late 1930's and early 1940's, mass vaccination was conducted in Brazil and various countries in Africa. Fox, J. P. et al (Fox, F. P. and Penna, H. A. (1943). Behavior of 17D yellow fever virus in Rhesus monkeys. Relation to substrain, dose and neural or extraneural inoculation. Am. J. Hyg. 38: 52-172) described the preparation of the vaccine from 17D virus substrains. These substrains differed in their passage history and they overlapped with regard to time of their use for inocula and/or vaccine production. The substitution of each one by the next was according to the experience gained during vaccine production, quality control and human vaccination in which the appearance of symptomatology led to the discontinuation of a given strain.

As mentioned before, the YF virus Asibi strain was subcultured in embryonic mouse tissue and minced whole chicken embryo with or without nervous tissue. These passages yielded the parent 17D strain at passage level 180, 17DD at passage 195, and the 17D-204 at passage 204. 17DD was further subcultured until passage 241 and underwent 43 additional passages in embryonated chicken eggs until the vaccine batch used for 17DD virus purification (passage 284). The 17D-204 was further subcultured to produce Colombia 88 strain which, upon passage in embryonated chicken eggs, gave rise to different vaccine seed lots currently in use in France (I. Pasteur, at passage 235) and in the United States (Connaught, at passage 234). Each of these 17D-204 strains was plaque purified in different cell lines, the virus finally amplified in SW13 cells and used for cDNA cloning and sequence analyses. These 17D-204 are named C-204 (Rice, C. M.; Lenches, E.; Eddy, S. R.; Shin, S. J.; Sheets, R. L. and Strauss, J. H. (1985). “Nucleotide sequence of yellow fever virus: implications for flavivirus gene expression and evolution”. Science. 229: 726-733) and F-204 (Despres, P.; Cahour, A.; Dupuy, A.; Deubel, V.; Bouloy, M.; Digoune, J. P.; Girard, M. (1987). “High genetic stability of the coding region for the structural proteins of yellow fever strain 17D”. J. Gen. Virol. 68: 2245-2247), respectively. The 17D-213 strain was derived from 17D-204 when the primary seed lot (S1 112-69) from the Federal Republic of Germany (FRG 83-66) was used by the World Health Organization (WHO) to produce an avian leukosis virus-free 17D seed (S1 213/77) at passage 237.

The 17D-213 at passage 239 was tested for monkey neurovirulence (R. S. Marchevsky, personal communication, see Duarte dos Santos et al. (Duarte dos Santos, C N, Post, P R, Carvalho, R, Ferreira I I, Rice C M and Galler, R. 1995. Complete nucleotide sequence of yellow fever virus vaccine strains 17DD and 17D-213. Virus Res. 35 :35-41) and was the subject of sequence analysis together with 17DD (at passage 284) and comparison to previously published nucleotide sequences of other YF virus strains (Duarte dos Santos et al, 1995; Asibi: Hahn, C. S.; Dalrymple, J. M.; Strauss, J. H. and Rice, C. M. (1987). “Comparison of the virulent Asibi strain of yellow fever virus with the 17D vaccine strain derived from it”. Proc. Natl. Acad. Sci. USA. 84: 2029-2033; 17D-204 strain, C-204: Rice. C. M.; Lenches, E. M.; Eddy, S. R.; Shin, S. J.; Sheets, R. L. and Strauss, J. H. (1985). “Nucleotide sequence of yellow fever virus: implications for flavivirus gene expression and evolution”. Science. 229: 726-733; F-204: Despres, P.; Cahour, R.; Dupuy, A.; Deubel, V.; Bouloy, M.; Digoutte, J. P. and Girard, M. (1987). “High genetic stability of the coding region for the structural proteins of yellow fever virus strain 17D”. J. Gen. Virol. 68: 2245-2247). FIG. 1 depicts the passage history of the original YF Asibi strain and derivation of YF 17D vaccine strains.

A total of 67 nucleotide differences, corresponding to 31 amino acid changes, were originally noted between the Asibi and 17D-204 genomic sequences (see Hahn, C. S. et al, 1987). The comparison between the nucleotide sequences of 17DD and 17D-213 substrains (see Duarte dos Santos et al, 1995) and the nucleotide sequence of 17D-204 substrain (see Rice et al, 1985) showed that not all changes are common and thus not confirmed as being 17D-specific. Therefore, the 17D-substrain specific changes observed are very likely not related to attenuation but may reflect differences in behaviour of these strains in monkey neurovirulence tests. Therefore 48 nucleotide sequence changes are likely associated with YF virus attenuation. These are scattered along the genome, 26 are silent mutations and 22 led to amino acid substitutions. The alterations noted in the E protein are important because it is the main target for humoral neutralizing response, i.e., it is the protein where hemagglutination and neutralization epitopes are located, and it mediates cell receptor recognition and cell penetration, therefore targeting the virus to specific cells. Importantly, E protein accumulate the highest ratio of nonconservative to conservative amino acid changes. Altogether, eleven nucleotide substitutions were observed in the E protein gene leading to 8 amino acid changes at positions 52, 170, 173, 200, 299, 305, 380 and 407 (respectively nucleotides 1127, 1482, 1491, 1572, 1870, 1887, 1965 and 2112 from the RNA 5′ end). Among these four are nonconservative changes and presumably would have a larger impact on protein structure and consequently function.

Alterations at amino acids 52 (G→R) and 200 (K→T) are located at the base of domain II in the 3-D structure proposed for the E protein of Flaviviruses (Rey, F. A.; Heinz F. X.; Mandl, C.; Kunz, C and Harrison, S. C. (1995). “The envelope glycoprotein from tick-borne encephalitis virus at 2A resolution”. Nature. 375: 291-298) which is conserved among Flaviviruses and contains cross-reactive epitopes as shown by Mandl, C. W. et al (Mandl, M. W.; Guirakhoo, F.; Holzmann, H.; Heinz, F. X. and Kunz, C. (1989). “Antigenic structure of the flavivirus envelope E protein at the molecular level using tick-borne encephalitis virus as a model”. J. Virol. 63: 564-571). This domain II is highly crosslinked by disulphide bonds and undergoes low pH transition which is related to exposing a strictly conserved and hydrophobic stretch of amino acids which are supposed to be involved in the fusion of the viral envelope to the endosome membrane.

Alterations at amino acids 170 and 173 in domain I of the E protein in the 3-D structure map very close to the position that a neutralization epitope was identified for tick-borne encephalitis (TBE) virus (see Mandl, C. W. et al, 1989). A mutation at position 171 of TBE virus E protein was shown to affect the threshold of fusion-activating conformational change of this protein and the 2 changes observed for YF 17D virus may be related to same phenomenon. It is conceivable that a slower rate of fusion may delay the kinetics of virus production and thereby lead to a milder infection of the host. Further evidence for the importance of this area comes from the work of Ryman et al (Ryman K D, Xie H, Ledger N, Campbell G A and Barrett A D T.1997. Antigenic variants of yellow fever virus with altered neurovirulence phenotype in mice. Virology 230, 376-380) showing that it encodes an epitope recognized by a wild-type-specific monoclonal antibody and reversion at this site may have contributed to added neurovirulence in the mouse model of a variant virus recovered from the 17D vaccine population.

Alterations at amino acids 299, 305, 380 and 407 are located in the domain III (see Rey, F. A. et al, 1995). This domain was suggested to be involved in viral attachment to a cellular receptor and consequently being a major determinant both of host range and cell tropism and of virulence/attenuation. The 4 amino acid changes reported for YF are located on the distal face of domain III. This area has a loop which is a tight turn in tick-borne encephalitis virus but contains 4 additional residues in all mosquito-borne strains. Because viruses replicate in their vectors, this loop is likely to be a host range determinant. This enlarged loop contains an Arginine-Glycine-Aspartic Acid (Arg-Gly-Asp) sequence in all 3 YF 17D vaccine strains. This sequence motif is known to mediate a number of cell interactions including receptor binding and is absent not only in the parental virulent Asibi strain but also in other 22 strains of YF wild type virus (Lepiniec L, Dalgarno L, Huong V T Q, Monath T P, Digoutte J P and Deubel V. (1994). Geographic distribution and evolution of yellow fever viruses based on direct sequencing of genomic DNA fragments. J. Gen. Virol. 75, 417-423). Such a fact suggests that the mutation from Threonine (Thr) to Arginine (Arg), creating a Arg-Gly-Asp motif, is likely to be relevant for the attenuated phenotype of the YF 17D strain. Consistently, Lobigs et al (Lobigs M, Usha R, Nesterowicz A, Marschall I D, Weir R C and Dalgarno L. 1990. Host cell selection of Murray Valley encephalitis virus variants altered at an RGD sequence in the envelope protein and in mouse neurovirulence. Virology 176, 587-595) identified a Arg-Gly-Asp sequence motif (at amino acid 390) which led to the loss of virulence of Murray Valley encephalitis virus for mice. At least for YF, however, it is not the only determinant as shown by van der Most et al (van der Most R G, Corver J, Strauss J H 1999. Mutagenesis of the RGD motif in the yellow fever virus 17D envelope protein. Virology 265, 83-95). It was suggested that the sequence in the RGD loop is critical for the conformation of E and minor changes in this region can have drastic effects on the stability of the protein. It is feasible, however, that such changes in structure and/or stability can affect the spectrum of cells infected by influencing the overall E protein structure and although that particular area would not be directly involved in the receptor binding. Since this loop is present in all mosquito-borne flaviviruses and most of them carry a sequence that is very similar to the 17D-RGD motif it is conceivable that mutations in this region in other flaviviruses will affect viral fitness.

The importance of residue 305 was implied from the findings by Jennings et al (1994) who noted that the 17D virus recovered from a human case of postvaccinal encephalitis had a E→K change at position 303 and was found to have increased neurovirulence for both mice and monkeys.

It is noteworthy that the development of infectious cDNA for Japanese encephalitis (JE) virus made by Sumiyoshi, H. et al (Sumiyoshi H, Hoke C H and Trent D W 1992. Infectious Japanese encephalitis virus RNA can be synthesized from in vitro-ligated cDNA templates. J. Virol. 66: 5425-5431) allowed the identification of a mutation (Lys for Glu) at amino acid 136 of the E protein which resulted in the loss of neurovirulence for mice (see Sumiyoshi, H.; Tignor, G. H. and Shope, R. E. (1996). “Characterization of a highly attenuated Japanese encephalitis virus generated from molecularly cloned cDNA”. J. Infect. Dis. 171: 1144-1151). This means that domain I is an important area which contains a critical determinant of JE virus virulence in contrast to most of the data obtained from the analyses of virulence for several other flaviviruses for which it is suggested that domain III would be the primary site for virulence/attenuation determinants.

For dengue type 2 virus Butrapet et al (2000) have suggested that attenuation determinants lie outside the structural area, namely, 5′ UTR, NS1 and NS3. Little is known about the molecular basis of viscerotropism, i.e. the ability of wild type YF virus to replicate and damage extraneural tissue, specially hepatic tissue, or the mutations responsible for the loss of this trait in the 17D virus. Further studies on 17D neurovirulence in the mouse model have suggested a complex genetic basis being most likely multigenic involving the structural and nonstructural proteins as well as the 3′ end nontranslated region, the latter would presumably restrict replication. Nevertheless, such analyses of the E protein provides a framework for understanding several aspects of flavivirus biology and suggests that it should be possible to engineer viruses for the development of new live flavivirus vaccines.

Being the major component of the virion surface, the envelope protein E plays a dominant role in eliciting neutralizing antibodies and the induction of a protective response. This has been conclusively demonstrated by active immunization of animals with defined subviral components and recombinant proteins and by passive protection experiments with E protein-specific monoclonal antibodies. Linear epitopes have been mapped using synthetic peptides and are found in areas of the glycoprotein predicted to be hydrophilic, however, the induction of neutralizing antibodies seems to be strongly dependent on the native conformation of E. A number of neutralizing sites have been inferred from studies with monoclonal antibody scape mutants and have been mapped onto the 3D structure. Consistent with the suggested topology of the dimer on the virion surface most of these sites are located on the externally accessible upper side of the subunit. The scattered distribution of these neutralization escape mutations over the entire subunit indicates that antibody binding to any of the structural domains can lead to virus neutralization.

The neutralization epitopes recognized by monoclonal antibodies are conformational since E protein denaturation abolishes binding. Moreover, monoclonal antibodies will only react with synthetic peptides if they recognize an epitope which is present on the denatured E protein. Since the dimeric subunit forms part of a as yet undefined lattice on the virion surface, it is likely that certain epitopes are composed of elements from different subunits.

The mechanisms of neutralization by these antibodies remains speculative but relevant to the strategy of epitope insertion described below is the hypothesis that domain II may be involved in fusion of the viral envelope with the membrane of the endosome, which occurs under acidic pH. Fusion requires conformational changes that affect several neutralization epitopes, primarily within central domain I and domain II. These changes are apparently associated with a reorganization of the subunit interactions on the virion surface, with trimer contacts being favored in the low pH form, in contrast to dimer contacts in the native form. Interference with these structural rearrangements by antibody binding represents one mechanism that may lead to virus neutralization (Monath and Heinz, 1996).

Low titer neutralizing activity and a significant degree of passive protection in mice has been observed by passive immunization with monoclonal antibodies against prM. This is due to a certain degree of partial cleavage of prM to form M by the furin in the Golgi system and thereby prM remains associated with the virus particle being an additional target for antibodies.

The NS1 protein, also known as the complement fixing antigen elicits an antibody response during the course of flavivirus infection in man. It exists as cell-associated and secreted forms and it has been shown that immunization of animals with purified NS1 or passive immunization of animals with monoclonal antibodies to it do elicit a protective immune response, the basis of which is still controversial. The primary immunological role of nonstructural proteins, except for NS1, seems to be targets for cytotoxic T cells. The specificity of T-cell responses to flaviviruses has been studied in human and mouse systems mainly with dengue and Japanese encephalitis serocomplex viruses. In the course of dengue infection in man, both CD4+, CD8− as well as the opposite CD4−, CD8+ T-lymphocytes response have been detected and characterized. In bulk cultures of CD4+ lymphocytes as well as with CD4+ cell clones obtained from a single individual which had been infected with dengue, different specific cross-reactivity patterns with several other flaviviruses is observed. Similar observations hold for CD8+ cells from infected humans and mice.

Antigenic determinants involved in cell mediated immunity have not yet been specifically localized in YF virus proteins as it has been for dengue and encephalitis virus such as MVE and JE. Such cytotoxic T cell determinants are found in all 3 structural and in the nonstructural proteins as well, specially in NS3. Some of these epitopes have been mapped to their primary sequence on the respective protein. Livingston et al (Livingston P G, Kurane I, Lai C J, Bray M, Ennis F A 1994. Recognition of envelope protein by dengue virus serotype-specific human CD4+ CD8− cytotoxic cell clones. J. Virol. 68, 3283-3288) reported the identification of several HLA class II-restricted CD4+ CTL clones from a human donor capable of mediating specific lysis of virus-infected cells. It has been suggested that CD4+ CTL may be important mediators of viral clearance especially during reinfection with the same serotype of virus.

Vaccination of humans with recombinant poxviruses expressing the structural proteins prM and E of Japanese encephalitis elicited CD4− CD8+ CTLs directed to the JE virus structural proteins although no specific epitopes were identified (Konish E, Kurane I, Mason P W, Shope R E, Kanesa-Thasan N, Smucny J J, Hoke C H, Ennis F A 1998. Induction of Japanese-encephalitis virus-specific cytotoxic T lymphocytes in humans by poxvirus-based JE vaccine candidates. Vaccine 16, 842-849).

More recently a JE virus E protein epitope recognized by JE-specific murine CD8+ CTLs has been reported. The epitope was found to correspond to amino acids 60-68 of the JE virus protein which are located in domain II (Takada K, Masaki H, Konishi E, Takahashi M, Kurane I 2000. Definition of an epitope on Japanese encephaltis virus envelope protein recognized by JEV-specific murine CD8+ cytotoxic T lymphocites. Arch. Virol. 145, 523-534). This epitope is located between strands a and b of domain II including two amino acid residues from each and the remaining of the epitope encompassing the intervening short loop. This area is exposed on the surface of the dimer.

Functional T-helper cell epitopes in the flavivirus E protein were identified by measuring B-cell response after immunization with synthetic peptides (Roehrig J T, Johnson A J, Hunt A R 1994. T-helper cell epitopes on the E glycoprotein of dengue 2 Jamaica virus. Virology 198, 31-38).

The capability to manipulate the genome of flaviviruses through infectious clone technology has opened new possibilities for vaccine development. This is so because virus can be recovered from complementary DNA by in vitro transcription and transfection of cultured cells with RNA, and these cDNAs corresponding to the complete viral genome allow introducing genetic modifications at any particular site of the viral genome. The pioneer study of Racaniello and Baltimore (Racaniello V R and Baltimore D 1981. Cloned poliovirus complementary DNA is infectious in mammalian cells. Science. 214, 916-919) first showed the feasibility to regenerate virus from cloned cDNA. In the patent U.S. Pat. No. 4,719,177, Racaniello and Baltimore described, in details, the production-of RNA viral cDNA by reverse transcribing viral RNA and inserting the resulting cDNA molecule into a recombinant DNA vector. The process was particularly concerned to the production of poliovirus double-stranded complementary DNA (ds cDNA). They found out that the transfected full-length poliovirus cDNA was itself infectious.

In addition, with the development of in vitro transcription systems (see Melton D A, Krieg P A, Rabagliati M R, Maniatis T, Zinn K and Green M R 1984. Efficient in vitro synthesis of biologically active RNA and RNA hybridization probes from plasmids containing a bacteriophage SP6 promoter. Nucl. Acids. Res. 12, 7035-7056), a much higher efficiency in synthesis of full length viral RNA, as compared to cDNA transcription in the cell, became possible. Furthermore, the development of improved transfection methodologies such as cationic liposomes and electroporation increased the efficiency of RNA transfection of cultured cells.

The construction and cloning of a stable full-length dengue cDNA copy in a strain of Escherichia coli using the pBR322 plasmid vector was described by Lai, C. J. et al (Lai C J, Zhao B, Hori H and Bray M. 1991. Infectious RNA transcribed from stably cloned full-length cDNA of dengue type 4 virus. Proc. Natl. Acad. Sci. USA. 88, 5139-5143). They verified that RNA molecules produced by in vitro transcription of the full-length cloned DNA template were infectious, and progeny virus recovered from transfected cells was indistinguishable from the parental virus from which the cDNA clone was derived. But, as mentioned in the Patent Application WO 93/06214, such an infectious DNA construct and RNA transcripts generated therefrom were pathogenic, and that the attenuated dengue viruses generated thus far were genetically unstable and had the potential to revert back to a pathogenic form overtime. To solve this problem, the Applicant proposed to construct cDNA sequences encoding the RNA transcripts to direct the production of chimeric dengue viruses incorporating mutations to recombinant DNA fragments generated therefrom. A preferred embodiment introduces deletions in the 3′ end noncoding region (Men R, Bray M, Clark D, Chanock R M, Lai C J 1996. Dengue type 4 virus mutants containing deletions in the 3′ noncoding region of the RNA genome: analysis of growth restriction in cell culture and altered viremia pattern and immunogenicity in rhesus monkeys. J. Virol. 70, 3930-3937; Lai C J, Bray M, Men R, Cahour A, Chen W, Kawano H, Tadano M, Hiramatsu K, Tokimatsu I, Pletnev A, Arakai S, Shameen G, Rinaudo M 1998. Evaluation of molecular strategies to develop a live dengue vaccine. Clin. Diagn. Virol. 10: 173-179; Whitehead S S, Men R H Lai C J, Murphy B R, Reynolds M J, Perreault J, Karron R A, Durbin A P, 2000. A live attenuated dengue virus type 4 vaccine candidate with a 30-nucleotide deletion in the 3′ end UTR is attenuated and immunogenic in humans. 19th Annual Meeting, American Society for Virology, pp. 125).

The construction of full-length YF 17D cDNA template that can be transcribed in vitro to yield infectious YF virus RNA was first described by Rice et al (Rice C M, Grakoui A, Galler R and Chambers T 1989. Transcription of infectious yellow fever RNA from full-length cDNA templates produced by in vitro ligation. The New Biologist 1: 285-296). Because of the instability of full-length YF cDNA clones and their toxic effects on Escherichia coli, they developed a strategy in which full-length templates for transcription were constructed by in vitro ligation of appropriate restriction fragments. Moreover, they found that the YF virus recovered from cDNA was indistinguishable from the parental virus by several criteria The YF infectious cDNA is derived from the 17D-204 substrain. Notwithstanding the YF virus generated from this YF infectious cDNA is rather attenuated, it cannot be used for human vaccination because of its residual neurovirulence, as determined by Marchevsky, R. S. et al (Marchevsky R S, Mariano J, Ferreira V S, Almeida E, Cerqueira M J, Carvalho R, Pissurno J W, Travassos da Rosa A P A, Simo\tilde{e}s M C, Santos C N D, Ferreira I I, Muylaert I R, Mann G F, Rice C M and Galler R 1995. Phenotypic analysis of yellow fever virus derived from complementary DNA. Am. J. Trop. Med. Hyg. 52, 75-80). Although these results showed the virus was not ideally attenuated for YF 17D vaccine it was the first demonstration for a flavivirus that it was possible to develop from a few micrograms of DNA template a whole seed lot under Good Manufacturing Practices (GMP) using current methodology for the production of YF vaccine.

Galler and Freire (U.S. patent application Ser. No. 09/058411) have approached the recovery of fully attenuated virus from YF cDNA by engineering a number of mutations into the original 17D-204 cDNA (Rice et al, 1989) based on the sequence of the 17DD substrain (Duarte dos Santos et al, 1995). This substrain has been used in Brazil for YF vaccine production since the late 1930's with excellent records of efficacy and safety. Here, virus was recovered from the genetically-modified cDNA template through the transfection of certified CEF cells under GMP (U.S. patent application Ser. No. 09/423517). Altogether 3 transfection lots were derived which gave rise to two primary and three secondary seed lots by further passaging in CEF cells with all the relevant quality controls for human vaccine production using this cell system. Average titer of formulated virus was 6.7log10 PFU/ml. Analysis of viral genetic stability was carried out by serial passaging in CEF cultures and studying several parameters such as plaque size, mouse neurovirulence and nucleotide sequence determination with satisfactory results.

Further nucleotide sequencing results on the chimeric 17D204/DD virus was obtained after viral reisolation from viremic monkey sera. There were no genomic changes (Galler R, Freire M S and JaborAV, unpublished). But YF 17D virus exists today as two main substrains used for vaccine production world wide, namely 17D-204 and 17DD, and differences in their sequences have been noted (Galler R, Post P R, Duarte dos Santos C N and Ferreira I I. 1998. Genetic variability among yellow fever virus 17D substrains. Vaccine 16, 1024-1028).

espite the overall genetic stability of YF 17D virus a major concern is the genetic stability of the foreign epitope since the virus does not need it and in fact its replication has been somewhat restricted. Although viral genetic variability is minimized by the use of seed lot system, to produce production-sized seeds at least 4 passages are necessary starting out from cDNA (U.S. Pat. No. 6,171,854).

The first aspect that has to be considered when using a given flavivirus cDNA backbone for the expression of heterologous proteins is whether one can indeed recover virus with the same phenotypic markers as originally present in the virus population that gave rise to the cDNA library. That is extremely applicable to YF 17D virus given the well known safety and efficacy of YF 17D vaccine.

In fact different technical approaches to constructing recombinant viruses based on a flavivirus, in particular YF 17D virus, are possible and will vary according to the region of the genome selected for insertion and to the antigen to be expressed. One major approach has been the creation of chimeric viruses through the exchange of prM/M/E genes as first established for DEN-4 virus chimeras (Lai et al, 1998, U.S. Pat. No. 5,494,671). The prM/M/E genes of dengue virus serotypes 1, 2 and 3 were inserted into the dengue 4 infectious clone resulting in chimeric virus with reduced virulence for mice and monkeys (Lai et al, 1998) This allows the removal of the major immunogens of the vector thereby reducing the criticism on previous inmmunity.

The same type of construction was made for tick-borne encephalitis (TBE) and Langat viruses (Pletnev A G, Bray M, Huggins J, Lai C J 1992. Construction and characterization of chimeric tick-borne encephalitis/dengue type 4 viruses. Proc. Natl. Acad Sci. USA. 89:10532-10536; Pletnev A G, Men R. 1998. Attenuation of Langat virus tick-borne flavivirus by chimerization with mosquito-borne flavivirus dengue type 4. Proc. Natl. Acad. Sci. USA. 95: 1746-1751) resulting in virus attenuated for mice.

Chambers et al (Chambers T J, Nestorowicz A, Mason P W, Rice C M 1999. Yellow fever/Japanese encefalitis chimeric viruses: construction and biological properties. J. Virol. 73, 3095-3101) have described the first chimeric virus developed with the YF 17D cDNA from Rice et al (1989) by the exchange of the prM/M/E genes with cDNA derived from JE SA14-14-2 and Nakayama strains of JE virus. The former corresponds to the live attenuated vaccine strain in use nowadays in China.

Guirakhoo et al (Guirakhoo F, Zhang Z X, Chambers T J, Delagrave S, Arroyo J. Barrett A D T, Monath T P 1999. Immunogenicity, genetic stability and protective efficacy of a recombinant, chimeric yellow fever-Japanese encephalitis virus (Chimerivax-JE) as a live, attenuated vaccine candidate against fever Japanese encephalitis. Virology 257: 363-372) Monath et al (Monath T P, Soike K, Levenbook I, Zhang Z X, Arroyo J, Delagrave S, Myers G, Barrett A D T, Shope R E, Rattterree M, Chambers T J, Guirakhoo F 1999. Recombinant, chimeric live, attenuated vaccine (Chimerivax) incorporating the envelope genes of Japanese encephalitis (SA14-14-2) virus and the capsid and nonstructural genes of yellow fever (17D) virus is safe, immunogenic and protective in nonhuman primates. Vaccine 17: 1869-1882) and WO98/37911 have brought it closer to vaccine development. Here, chimeric virus was recovered after transfection of certified FRhL cells with 5 additional passages of the virus to produce seed lots and experimental vaccine lot (5th passage) all under GMP in certified cells. Virus yields in this cell system were not provided.

Chimeric virus retained nucleotide/amino acid sequences present in the original SA14-14-2 strain. This vaccine strain differs, in prM/M/E region, from the parental virus in 6 positions (E-107; E138; E176: E279; E315; E439). Mutations are stable across multiple passages in cell culture (Vero) and mouse brain but not in FRhL cells. Despite previous data on the genetic stability of such virus, one of the 4 changes in the E protein related to viral attenuation had reverted during the passaging to produce the secondary seed.

In a dose-response study neutralizing antibodies specific for prM/M/E were elicited in all groups of monkeys with different doses even with as little as 100 PFUs and conferred full protection against IC challenge with wild type JE. However, the lower the chimeric virus dose the more residual histopathological changes were noted in the SNC after IC challenge with wild type JE virus.

The first chimeric 17D/dengue virus developed (Guirakhoo F, Weltzin R, Chambers T J, Zhang Z X, Soike K, Rattterree M, Arroyo J, Georgakopoulos K, Cataian J, Monath T P 2000. Recombinant chimeric yellow fever-dengue type 2 virus is immunogenic and protective in nonhuman primates. J. Virol. 74, 5477-5485) involved prM/M/E gene replacement (fusion at the signalase cleavage site) with a den2 cDNA. All virus regeneration and passaging was done in Vero PM cells (a cell bank from Pasteur-Merieux) allegedly certified for live vaccine virus production. Recombinant virus retained the original den2 prM/M/E sequences even after 18 serial passages in Vero cells but some variation was noted in YF genes. Phenotypic analysis of chimeric 17D/den2 virus showed it does not kill mice even at high doses (6.0 log10 PFU) in contrast to YF 17D which kills nearly 100% at 3.0 log10 PFU. Antibody response and full protection was elicited by the 17D-DEN2 chimera in both YF immune and flavivirus-naive monkeys. In a dose response study even at the lowest dose (2.0 log10 PFU) chimeric virus replicated sufficiently to induce a protective neutralizing antibody response as no viremia was detected in these animals after challenge with a wild type dengue 2 virus.

Although YF 17D virus is known to be more genetically stable than other vaccine viruses, such as poliovirus, given the extremely low number of reports on adverse events following vaccination, a few mutations have been detected occasionally when virus derived from humans were sequenced (Xie H, Cass A R, Barrett A D T 1998. Yellow fever 17D vaccine virus isolated from healthy vaccinees accumulates few mutations. Virus Research 55:93-99). Guirakhoo et al have reported a few changes in the YF moiety of chimeric 17D/dengue 2 virus which had been passaged up to 18 times in cell culture.

Galler et al (in preparation) have also developed a similar chimeric 17D-DEN-2 virus. However, the 17D backbone was genetically modified (U.S. Pat. No. 6,171,854). These viruses were characterized at the genomic level by RT/PCR with YF/Den-specific primers and nucleotide sequencing over fusion areas and the whole DEN2-moieties. The polyprotein expression/processing was monitored by SDS-PAGE analysis of radiolabeled viral proteins immunoprecipitated with specific antisera, including monoclonal antibodies. Recognition of YF and DEN-2 proteins by hiperimmune antisera, and monoclonal antibodies was also accomplished by viral neutralization in plaque formation reduction tests and indirect immunofluorescence on infected cells. The growth of recombinant viruses was examined in several cell substrates such as Vero, LLC-MK2, C6/36, MRC5, and CEF. Only YF virus grew in all of them to high titers but the chimeric viruses failed to replicate in the vaccine-production certified cells (CEF and MRC5) similarly to DEN-2 virus. Analysis of viral virulence was performed by intracerebral inoculation of mice (10³ PFU) and the chimeric viruses turned out to be more attenuated in this system than the YF 17D virus. With regard to the immunogenicity, studies in the mouse model indicate the chimeric virus does induce a protective response against an otherwise lethal dose of mouse neurovirulent DEN-2 New Guinea C virus.

One alternative to develop YFV 17D as a vector for heterologous antigens is the expression of particular epitopes in certain regions of the genome. The feasibility of this approach was first demonstrated for poliovirus (reviewed in Rose C S P, Evans D J 1990 Poliovirus antigen chimeras. Trends Biotechnol. 9:415-421). The solution of the three-dimensional structure of poliovirus allowed the mapping of type-specific neutralization epitopes on defined surface regions of the viral particle (Hogle J M, Chow M & Filman D J (1985). Three-dimensional structure of poliovirus at 2.9 resolution. Science 229:1358-1365). One of the surface loops of the VP1 protein was used for the insertion of type 3 epitope which was recognized by primate antisera to poliovirus type 3 showing that the chimera was not only viable but also that the inserted epitope was presented with the same conformation as in the surface of the type 3 virus (Murray M G, Kuhn R J, Arita M, Kawamura N, Nomoto A & Wimmer E (1988) Poliovirus type 1/type 3 antigenic hybrid virus constructed in vitro elicits type 1 and type 3 neutralizing antibodies in rabbits and monkeys. Proc.Natl.Acad.Sci. USA 85:3203-3207). However, to argue strongly against it is the observation that the same site was used for the insertion of different epitopes of hepatitis A virus but the immunogenicity of the inserted peptides was very poor (Lemon S M, Barclay W, Ferguson M, Murphy P, Jing L, Burke K, Wood D, Katrak K, Sangar D, Minor P D & Almond J W (1992). Immunogenicity and antigenicity of chimeric picornaviruses which express hepatitis A virus (HAV) peptide sequences: evidence for a neutralization domain near the amino terminus of VP1 of HAV. Virology 188:285-295).

Influenza viruses are also well studied from the structural view and 3D structures are available for both hemagglutin and neuraminidase viral proteins. Li et al (Li S, Polonis V, Isobe H, Zaghouani H, Guinea R, Moran T, Bona C, Palese P 1993. Chimeric influenza virus induces neutralizing antibodies and cytotoxic T cells against human immunodeficiency virus type 1. J. Virol. 67: 6659-6666) have described insertion of HIV epitope into a loop of antigenic site B of influenza virus and the generation of specific B and T cell responses to the epitope.

London et al (London S D, Schmaljohn A L, Dalrymple J M & Rice C M (1992) Infectious enveloped RNA virus antigenic chimeras. Proc.Natl.Acad.Sci. USA 89:207-211) developed chimeric Sindbis virus expressing a single well-defined neutralizing epitope of Rift Valley fever virus inserted by random mutagenesis. Insertion sites, permissive for recovery of viruses with growth properties similar to the parental virus, were found in one of the virion glycoproteins. For these chimeras the epitope was expressed at the virion surface and stimulated a partially protective immune response to RFV infection of mice.

De Vries et al (de Vries A A F, Glaser A L, Raasman M J B, de Haan C A M, Sarnataro G J G, Rottier P J M 2000. Genetic manipulation of equine arteritis virus using full-length cDNA clones: separation of overlapping genes and expression of a foreign epitope. Virology 270, 84-97) have engineered equine arteritis virus, also a positive-stranded RNA virus with a genome similar in size to YF 17D but with different replication strategy. Insertion of a nonapeptide epitope recognized by an MHV-specific monoclonal antibody with a functional O-glycosylation site resulted in recombinant virus expressing both properties of the epitope. However, growth of the virus was clearly reduced as compared to the parental virus although 3 serial passages did not result in the loss of the insert.

Bendahmane et al (Bendahmane M, Koo M, Karrer E, Beachy R N 1999. Display of epitopes on the suface of tobacco mosaic virus: impact of charge and isoelectric point of the epitope on virus-host interactions. J. Mol.Biol. 290, 9-20) have described the creation of chimeric tobacco mosaic virus expressing defined epitopes of rabies and murine hepatitis virus. The foreign epitopes were expressed on the carboxi terminus of the viral coat protein, the stucture and biophysical properties of which is well established. Although both insertions gave rise to viable viruses the generation of lesions in plants infected by either virus differed. It was observed that the isoelectric point (pI) of the epitope affected the overall hybrid coat protein pI/charge and that was important for successful epitope display. It was also noted the lack of tolerance to positively charged epitopes on the surface of TMV.

A number of other studies employing virus-like particles to express defined foreign protein epitopes have been described as they seem to be able to potentiate the immunogenicity of foreign epitopes presented on their surface. Heal et al (Heal K G, Hill H R, Stockley P G, Hollingdale M R, Taylor-Robinson A W 2000. Expression and immunogenicity of a liver stage malaria epitope presented as a foreign peptide on the surface of RNA-free MS2 bacteriophage capsids. Vaccine 18, 251-258) have used the coat protein of bacteriophage MS2 to express foregin epitopes based on a β-hairpin loop at the N-terminus of this protein which forms the most radially distinct feature of the mature capsid. A chimeric capsid expressing a Plasmodium liver-stage antigen epitope (LSA-1) stimulated in mice a polarized Th-1 response similar to the human response to this antigen in nature.

Of interest is also the study by Sedlik et al (Sedlik C, Sarraseca J, Rueda P, Leclerc C, Casal J I 1995. Immunogenicity of poliovirus B and T cell epitopes presented by hybrid porcine parvovirus particles. J. Gen. Virol. 76, 2361-2368). They have inserted poliovirus T and B cell epitopes into the single-stranded DNA genome of canine parvovirus. Insertions were made at the N terminus of the viral VP2 on the particle structure. There was no antibody response to the epitope whereas a T cell response could be observed in vitro for the expressed T cell epitope. That suggested the N-terminus is not an adequate site for the expression of humoral epitopes. Rueda et al (Rueda P, Hurtado A, Barrio M, Torrecuadrada J L M, Kamstup S, Leclerc C, Casal J I 1999. Minor displacements in the insertion site provoke major differences in the induction of antibody responses by chimeric parvovirus-like particles. Virology 263, 89-99) have extended these studies with canine parvovirus and inserted a poliovirus humoral epitope into the 4 loops of the viral VP2 carboxi terminus on the particle structure. Only loop 2 allowed the generation of antibodies to the epitope but not poliovirus neutralizing antibodies. That was accomplished by inserting the epitope at adjacent amino acids suggesting that minor displacements at the insertion site cause dramatic changes in the accessibility of the epitope and the induction of antibody responses.

Chimeric hepatitis B virus core particles carrying hantavirus epitopes have been described by Ulrich et al (Ulrich R, Koletzki D, Lachman S, Lundkvist A, Zankl A, Kazaks A, Kurth A, Gelderblom H R, Borisova G, Meisel H, Krueger D H 1999. New chimaeric hepatitis B virus core partciles carrying hantavirus (serotype Puumala) epitopes: immunogenicity and protection against virus challenge. J. Biotechnol. 73, 141-153). Insertion of as much as 45 and 114 amino acids of hantavirus capsid protein resulted in protection of natural hosts to the virus.

The formation of chimeric virus-like partciles with hamster polyoma virus major capsied protein has been describe by Gedvilaite et al (Gedvilaite A, Froemmel C, Sasnaukas K, Micheel B, Oezel M, Behrsing O, Stanilius J, Jandrig B, Scherneck S, Ulrich R 2000. Formation of immunogenic virus-like particles by inserting epitopes into surfac-exposed regions of hamster polyomavirus major capsid protein. Virology 273, 21-35). They have predicted from the 3D-structure 3 areas that would be surface exposed and expression of a 5-amino acid hepatitis B virus capsid epitope resulted in the recognition by a monoclonal antibody to the epitope.

For Flaviviruses, neutralizing epitopes to other viral agents could be inserted in regions of the viral envelope where one detects genetic variability by nucleotide sequencing. Such an approach was first tested after changing 2 amino acids in the envelope protein gene of the YF infectious cDNA by the corresponding amino acid sequence of Murray Valley encephalitis virus (MVE) which was previously characterized by monoclonal antibody as a neutralizing MVE epitope The chimera, however, was not viable suggesting that particular area of the E protein (amino acids 192-193 from the amino terminal) is critical for YF virus viability (R. Weir and C. Rice, pers. commun.).

In short, to obtain a Flavivirus-based vaccine virus expressing foreign antigens for the development of new live vaccines using recombinant DNA techniques, it is necessary, cumulatively:

(1) to design strategies that allow the introduction of foreign antigens without compromising the structure and function of the virus;

(2) to assure that the infectious cDNA construct and RNA transcripts generated therefrom give rise to virus which is not pathogenic, does not have the potential to revert to a pathogenic form; and that the foreign antigen sequence is genetically stable once integrated into the viral genome;

(3) the Flavivirus generated from cloned cDNA, in addition to being attenuated should retain its immunological properties and present the expressed foreign antigen such that it elicits the appropriate immune response.

### SUMMARY OF THE INVENTION

It is an object of the present invention to provide a safe and effective Flavivirus vaccine virus obtained from a cloned cDNA having phenotypic characteristics such as attenuation and immunogenicity, and which should also stably express and elicit an immune response to foreign antigens.

In one embodiment, the present invention relates to a method for the production of Flavivirus as a vector for heterologous antigens comprising the introduction and expression of foreign gene sequences into an insertion site at the level of the envelope protein of any Flavivirus, wherein the sites are structurally apart from areas known to interfere with the overall flavivirus E protein structure and comprising: sites that lie on the external surface of the virus providing accessibility to antibody; not disrupt or significantly destabilize the three-dimensional structure of the E protein and not interfere with the formation of the E protein network within the viral envelope.

In another embodiment the present invention is related to a strategy that allows introducing foreign gene sequences into the fg loop of the envelope protein of YF 17D virus and other flaviviruses.

Another embodiment of the present invention relates to a new version of YF infectious cDNA template that is 17DD-like and which resulted of insertion of malarial gene sequences.

In another embodiment of the present invention, there is provided new YF plasmids which have the complete sequence of the YF infectious cDNA and malarial gene sequences.

Another embodiment of the present invention are Flavivirus as a vector for heterologous antigens wherein the Flavivirus is obtainable according to the method herein described.

In another embodiments there is provided recombinant YF viruses which are regenerated from a YF infectious cDNA and express different malarial epitopes.

### BRIEF DESCRIPTION OF THE DRAWINGS

FIG. 1 illustrates the passage history of the original YF Asibi strain and derivation of YF 17D vaccine strains.

FIG. 2 shows the sequence alignment of the soluble portions of the Envelope proteins from tick-borne encephalitis virus (tbe), yellow fever virus (yf), japanese encephalitis virus (je) and Dengue virus type 2 (den2).

FIG. 3: shows a schematic representation of the CS protein of Plasmodium sp..

FIG. 4: displays the structure of the plasmid pYF17D/14.

FIG. 5: shows the structure of the plasmid pYFE200.

FIG. 6: shows the sequence alignment between the and yf, but with the introduction of an insertion sequence (highlighted in bold and underlined) between residues 199 and 200 of yf, located in the loop between β-strands f and g. As in FIG. 2, the alignment shown is that used for model building of the modified yf E protein and deliberate misalignments are shown shaded. Elements of secondary structure are shown as horizontal bars between the two sequences.

FIG. 7: sets forth two views of the modelled yf E protein including the SYVPSAEQI insertion sequence within the fg loop. The domains are coloured individually, domain I (red), domain II (yellow) and domain III (blue). In the upper panel the E protein dimer is seen perpendicular to the viral membrane and in the lower panel is viewed within the membrane plane, perpendicular to the long axis of the dimer. The insertion site (in cyan) lies close to the proximal interface between the two constituent monomers of the dimer and can be seen to be partially buried.

FIG. 8: sets forth the superposition of ten models of the YF E protein including the insertion sequence GG(NANP)₃GG within the fg loop. In each model the insertion sequence is shown in a different color while the remainder of the structure is shown in green. The great diversity in conformations for the loop, while essentially preserving the rest of the structure, indicates that the large volume of space available to the insertion peptide.

FIG. 9: shows the molecular surface of the YF E protein dimer for one of the ten models of FIG. 8. In the upper panel, the blue and red dots indicated on each monomer, represent the entrance and exit to the insertion peptide. In the middle panel the two-residue N-(blue) and C-terminal (red) glycine spacers are shown, indicating their role in lifting the (NANP)₃ sequence above the molecular surface. In the lower panel the (NANP)₃ insertion is shown in green.

FIG. 10: sets forth an indirect immunofluorescence assay using a monoclonal antibody directed to (NANP)₃ repeat.

FIG. 11: displays a SDS-PAGE gel of the 17D/8 virus obtained by immunoprecipitation of metabolic labeled viral proteins.

FIG. 12: illustrates the comparative plaque size analysis among YF 17D/8 virus, YF17D/14 and YF17D/G1/2-derived virus.

FIG. 13: shows viral growth curves in CEF (15a) and VERO cells (15b).

FIG. 14: sets forth the size of virus plaques formed on Vero cell monolayers after serial propagation of the viruses in Vero and CEF cell cultures.

FIG. 15: shows the comparative growth curves of the different recombinant viruses in Vero cells.

FIG. 16: shows the plaque size analysis of the different recombinant YF viruses.

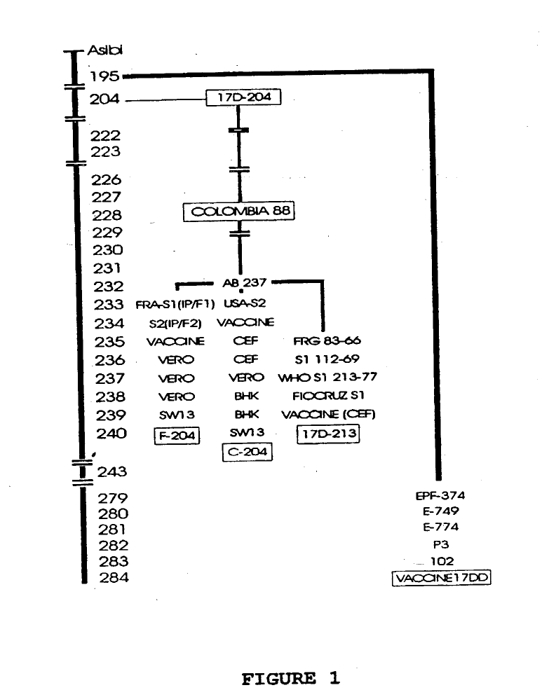

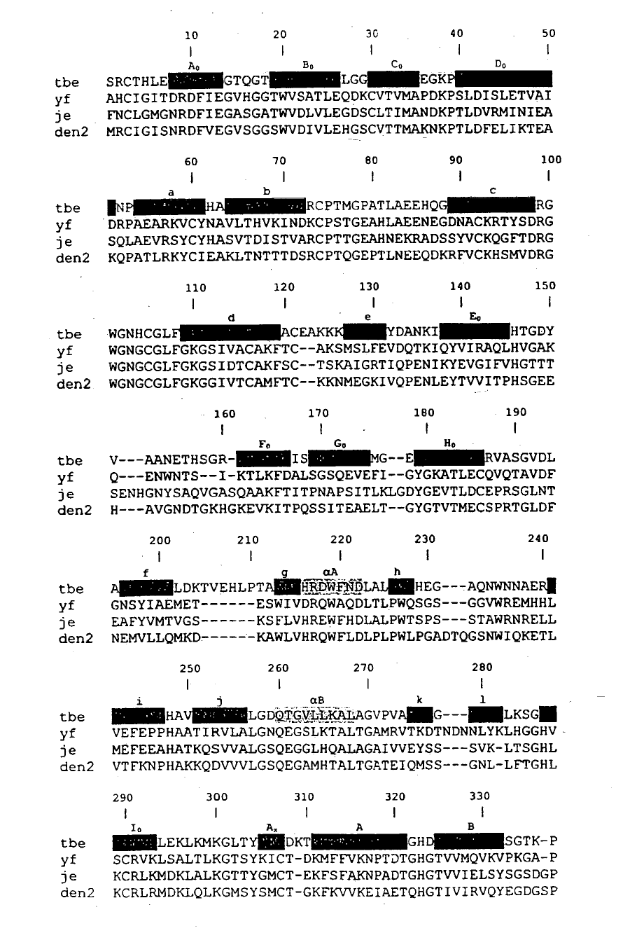

### DETAILED DESCRIPTION OF THE INVENTION

For many diseases, the ideal vaccine is a live attenuated derivative of the pathogen, which induces strong, long-lasting protective immmune responses to a variety of antigens on the pathogen without causing illness. Development of such vaccine is often precluded by difficulties in propagating the pathogen, in attenuating it without loosing immunogenicity and ensuring the stability of the attenuated phenotype. One alternative is the use of known attenuated microorganisms for the expression of any antigen of interest.

The ability to genetically engineer animal viruses has changed the understanding of how these viruses replicate and allowed the construction of vectors to direct the expression of heterologous proteins in different systems. DNA viruses such as SV40, Vaccinia, adenoviruses and herpes have been used as vectors for a number of proteins. More recently RNA viruses, both positive and negative stranded, (Palese P 1998. RNA virus vectors: where are we and where do we need to go? Proc. Natl. Acad. Sci. USA 95,12750-2) have also become amenable to genetic manipulation and are preferred vectors as they lack a DNA phase ruling out integration of foreign sequences into chromosomal DNA and do not appear to downmodulate the immune response as large DNA viruses do (eg. vaccinia and herpes).

Flaviviruses have several characteristics which are desirable for vaccines in general and that has attracted the interest of several laboratories in developing it further to be used as a vector for heterologous antigens. Particularly for YF17D virus, these characteristics include well-defined and efficient production methodology, strict quality control including monkey neurovirulence testing, long lasting immunity, cheapness, single dosis, estimated use is over 200 million doses with excellent records of safety (only 21 cases of post-vaccinal encephalitis after seed lot system implementation in 1945 with an incidence in very young infants (9 months) of 0.5-4/1000 and >9 months at ⅛ million).

The fact that the 3-D structure for the flavivirus E protein is available (Rey et al, 1995) would support the approach first used for poliovirus by examining the 3D-structure and selecting sites for insertion which are less likely to interfere with the overall E protein structure. The major concern about inserting epitopes into flaviviruses E protein, in particular into the YF 17D E protein, relates to the fact that this protein is the main target for humoral neutralizing response, it is the protein where hemagglutination and neutralization epitopes are, and it is the protein that mediates cell receptor recogniton and cell penetration, therefore targeting the virus to specific cells. By inserting a new epitope somewhere in the E protein of a given flavivirus one or more of these properties could be changed unless the analysis of the 3D structure allows the identification of potential insertion sites structurally apart from areas known to mediate one or more of these processes.

Regarding the Tick-borne encephalitis virus E protein, two distinct crystal forms of its soluble fragment were obtained by Rey et al. (Rey et al., 1995). In both, the E protein shows a similar dimeric arrangement in which two monomers are related by a molecular twofold axis which is crystallographic in one crystal form and non-crystallographic in the other. The repeated appearance of the same dimer in both cases suggests that this is not an artifact of crystallization but represents the true oligomeric arrangement of the E protein as inserted into the viral envelope at neutral pH. The dimer presents an elongated flattened structure with overall dimensions of approximately 150×55×30 Å. Its shortest dimension lies perpendicular to the viral membrane and the whole structure presents a mild curvature yielding an external surface which is somewhat greater than the corresponding internal surface. This curvature may aid in the closure of the spherical viral particle. Each monomer is composed of three domains; domain I (the central domain), domain II (the dimerization domain) and domain III (the immunoglobulin-like receptor binding domain), all of which are dominated by β-sheet secondary structure. Domain I is discontinuous, being composed of three separate segments of the polypeptide chain, and is dominated by an up-and-down eight-stranded β-barrel of complex topology. Domain II is responsible for the principal interface between the two monomers proximal to the two-fold axis and is formed by the two segments of the polypeptide chain which divide domain I. It is an elongated domain, heavily crosslinked by disulphide bridges and composed principally of two structural components; 1) a five-stranded anti-parallel β-sheet onto one side of which pack the only two α-helices of the structure and 2) a β-sandwich made up of a three-stranded β-sheet packed against a β-hairpin. This β-sandwich sub-domain includes the fusion peptide believed to be important for the fusogenic activity of the virus. It nestles into a cavity formed by the interdomain contacts between domains I and III of the opposite monomer and therefore forms part of a second interface region between the two monomers which lies more distal to the twofold axis to that mentioned above. Domain III is continuous and presents a somewhat modified C-type immunoglobulin (Ig) fold. Using the conventional nomenclature for such folds (Bork P, Holm L, Sander C, 1994 The immunoglobulin fold, J. Mol. Biol. 242, 309-320), the C, F and G strands of this domain face outwards from the monomer and represent a region critical in the determination of host range and cell tropism and is probably therefore fundamental for cell attachment. The opposite face of the Ig-like domain forms the interface with domain I, and together with regions from the β-sandwich sub-domain of the opposite monomer, is important in forming the dimer interface distal to the twofold axis. This interface is further protected by the carbohydrate moiety present on domain I.

The β-strands from domain I are named A₀ to I₀, those from domain II named a to 1 and those from domain III named A to G, in all cases labeled consecutively from the N-terminus (in domain III a distortion of the typical C-type Ig-fold leads to the creation of additional strands Aₓ, Cₓ and Dₓ). With the exception of the two short α-helices of domain II, all connections between the β-strands of a given domain as well as the linkers which lead from one domain to another are either β-turns or loops which vary greatly in length. In general terms all such loops are either buried within the structure (inaccessible to solvent) or exposed on one or more of the internal, external and lateral surfaces of the dimer.

On exposure to low pH the network of E protein dimers on the viral surface must rearrange into trimers. This must envolve large alterations to the monomer-monomer contacts and possibly also to the relative domain orientations within a given subunit, if not to internal reorganization of the tertiary structure of the domains themselves. In participating in both proximal and distal contacts, domain II is likely to suffer the greatest changes, consistent with the fact that the binding of monoclonal antibodies to this domain is strongly affected by the dimer to trimer transition (Heinz F X, Stiasny K, Puschnerauer G, Holzmann H, Allison S L, Mandl C W, Kunz C 1994 Structural-Changes And Functional Control Of The Tick-Borne Encephalitis-Virus Glycoprotein-E By The Heterodimeric Association With Protein prM Virology 198, 109-117).

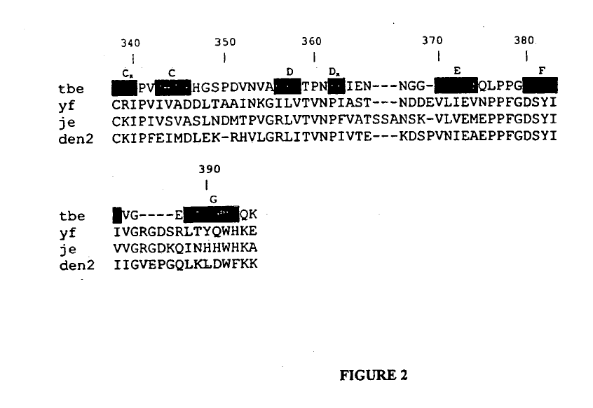

For the design of insertions of epitopic peptides into the E protein of a given Flavivirus and the subsequent evaluation of their viability the inventors of the present invention developed the following strategy. Initially it was necessary to produce a three-dimensional model for the E protein of a selected Flavivirus. The sequence of the yellow fever 17DD strain was used for this purpose and its alignment with that of tick-borne encephalitis (tbe) virus was generated initially with the program MULTALIGN (Barton G J, Sternberg M J E, 1987, A Strategy For The Rapid Multiple Alignment Of Protein Sequences—Confidence Levels From Tertiary Structure Comparisons J Mol Biol 198, 327-337) and subsequently with reference to the 3D structure of the latter, such that all insertions and deletions were restricted to stereochemically reasonable positions. A final alignment, including sequences from japanese encephalitis (strain JaOArS982) and Dengue type 2 (strain PR-159), is shown in FIG. 2 and is significantly different in several important respects from that given by Rey et al, 1995. The most important differences, which are relevant to the subsequent insertion design are now described.

An insertion of two residues (122 and 123 in tbe) is introduced in tbe leading to a complete readjustment of the alignment up to the region of the glycosylation site between β-strands E₀ and F₀. On comparing the tbe and yellow fever (yf) sequences within the region 120 to 150 (tbe sequence numbering), the alteration leads to an increase in the number of sequence identities from 2 to 8. A similar improvement is observed for japanese encephalitis (je) and an increase from 1 to 10 identities occurs in the case of Dengue 2 (d2). Besides the clear improvement in sequence identity, the new alignment accommodates the two residue insertion (122 and 123 in tbe) in a surface loop (between β-strands d and e).

A deletion of one residue prior to strand f in yf and d2 is closed and transferred to the large deletion between β-strands f and g. The deletion in this region of the alignment given in FIG. 2 is thus 6 residues in length for both yf and d2, as it is in je, when compared to tbe.

The asparagine/aspartic acid rich segment of yf (residues 269 to 272) becomes an insertion between β-strands k and l of domain II.

The sequence alignment was used to generate 10 models for the yf E protein dimer using satisfaction of spatial restraints derived from the tbe dimeric structure employing the program MODELLER (Sali A, Blundell, T L 1993, Comparative model building by satisfaction of spatial restraints. J. Mol. Biol. 234, 779-815). For each model a default coordinate randomization in cartesian space of 4 Å was employed prior to model optimization using the Variable Target Function Method (Braun W, Go N 1985 Calculation of protein conformations by proton proton distance constraints—a new efficient algorithm J Mol Biol 186, 611-626) and Simulated Annealing. Deliberate misalignment of residues surrounding insertions and deletions was used in order to relax the homology constraints of these residues, permiting the insertion/deletion while simultaneously maintaining acceptable stereochemistry.

The model which presented the lowest pseudo-energy, given by −1n(P), where P is the MODELLER molecular probability density function, also presented excellent overall stereochemistry, yielding for example a PROCHECK (Laskowski R A, Macarthur M W, Moss D S, Thornton J M, 1993 Procheck—A Program To Check The Stereochemical Quality Of Protein Structures, Journal Of Applied Crystallography 26: 283-291) G-factor of −0.1, equivalent to a crystal structure of better than 1.5 Å resolution. The model was also evaluated using the method of Eisenberg (Eisenberg D, Luthy R, Bowie J U, 1997, VERIFY3D: Assessment of protein models with three-dimensional profiles Method Enzymol 277: 396-404; Bowie J U, Luthy R, Eisenberg D A, 1991, Method to Identify Protein Sequences that fold into a Known 3-Dimensional Structure Science 253, 164-170 Luthy R, Bowie J U, Eisenberg D, 1992 Assessment Of Protein Models With 3-Dimensional Profiles Nature 356, 83-85), presenting a VERIFY_{—}3D score of 348, close to the expected value of 361 for a protein of 786 residues (in the dimer) and well above the acceptability threshold of 162. Furthermore the normality of the model was assessed by atomic contact analysis using the WHATIF overall quality score (Vriend G, 1990. What If—A Molecular Modeling And Drug Design Program J Mol Graphics 8 52-57; Vriend G, Sander C, 1993 Quality-Control Of Protein Models—Directional Atomic Contact Analysis J Appl Crystallogr 26 47-60) which gave a value of −0.964, showing the model to be reliable.

The model for the yf E protein shows a slightly reduced contact area between subunits compared with tbe (1,242 Å² per monomer compared with 1,503 Å²), partly due to the reduced size of the fg loop which makes intersubunit contacts via His208 in tbe. There is a subsequent reduction in interdomain hydrophobic contacts as detected by LIGPLOT (Wallace A C, Laskowski R A, Thornton J M, 1995. Ligplot—A Program To Generate Schematic Diagrams of Protein Ligand Interactions Protein Eng 8 127-134). These are compensated by the appearance of two potential intersubunit salt bridges (between E201 and R243 and between R263 and E235) which appear in several of the ten models and which are absent, due to amino acid substitutions, in tbe. These salt bridges presumably aid in stabilizing the yf dimer. The contact made between the fusion peptide of the cd loop in domain II and the cavity between domains I and III of the opposite subunit is essentially retained in the yf E protein.

The model for the yf E protein together with the sequence alignment was used to select potential insertion sites for heterologous B and T cell epitopes. In both cases such an insertion site should 1) not disrupt or significantly destabilize the three-dimensional structure of the E protein; 2) not interfere with the formation of the E protein network within the viral envelope; 3) lie on the external surface of the virus such that it is accessible to anti-body. Although this criterion may not be strictly obligatory for T-cell epitopes it remains appropriate as sites on the internal surface may interfer with viral assembly. 4) The site should preferably present evidence that sequence length variation is permissible from the differences observed between different flaviviruses (ie. the site should show natural variance). 5) In the case of sites which present sequence length variation, preferably yf should present a smaller loop in such cases.

The first criterion limits insertion sites to loops and turns between elements of secondary structure. The second and third eliminate sites on the internal and lateral surfaces of the dimer and those that are buried. Of the remaining possible insertion sites, the following can be said. The loop between Dₒ and a represents an interdomain connection and shows little structural variability. That between loops c and d represents the fusion peptide, is partially buried and highly conserved. That between d and e shows little structural variation and includes a ½-cystine residue which is structurally important. That between E₀ and F₀ includes the glycosylation site in tbe and is a potential insertion site as it shows great structural variability and is highly exposed. That between G₀ and H₀ may be partially involved in lateral contacts, is a small β-turn and shows little structural variation. That between f and g presents all of the desirable characteristics in that it is six residues shorter in yf, je and d2 compared with tbe and is externally exposed. That between k and l is a potential site as it shows an asparagine rich insertion in yf, which may accommodate asparagine-rich epitopic B-cell sequences as described below. That between B and Cₓ is a possible site but may form part of the lateral surface. That between Dₓ and E shows structural variation and presents an asparagine-rich sequence in yf which may accommodate asparagine-rich epitopes. From the above, the most promissing insertion site is that between β-strands f and g which form part of the five-stranded anti-parallel β-sheet of domain II. The large deletion of six residues in this loop in yf compared to tbe, leaves space at the dimer interface for a large insertion without creating steric hindrance. Besides the fg loop another promising insertion site is the E₀F₀ as it shows great structural variability and is highly exposed. Although not wishing to be bound by any particular theory, it is postulated that the presence of one or more glycine residues immediately flanking the inserted epitope is advantageous in introducing conformational flexibility to the epitope in its subsequent presentation.

One alternative of the present invention to develop flavivirus in general as a vector for heterologous antigens is the insertion and expression of particular antigens, including epitopes, into sites structurally apart from areas known to interfere with the overall flavivirus E protein structure, specially into the fg loop or the E₀F₀ loop of a given flavivirus E protein. The foreign inserted antigen, including epitope, may vary widely dependent on the immunogenic properties desired in the antigen. For example, the foreign inserted antigen may include antigens from protozoa such as malaria, from virus such as yellow fever, dengue, Japanese encephalitis, tick-borne encephalitis, fungi infections and others. Additionally, the maximum lenght of the antigen/epitope will depend on the fact that it would not compromise the structure and the function of the flavivirus envelope.

More particularly, one strategy described here is the insertion of malarial gene sequences into the fg loop of YF17D E protein. While comparatively short sequences having only a few amino acid residues may be inserted, it is also contemplated that longer antigens/epitopes may be inserted. The maximum lenght and the nature of the antigen/epitope will depend on the fact that it would not compromise the structure and the function of the yellow fever virus envelope.

Malaria remains one of the most important vector-borne human diseases. The concept that vaccination may be a useful tool to control the disease is based mainly on the fact that individuals continually exposed to infection by the parasitic protozoan eventually develop immunity to the disease.

The life cycle of the malaria parasite is complex, the several stages in humans are morphologically and antigenically distinct, and immunity is stage specific. It is only now becoming possible to define the full pattern of parasite gene expression in each stage.

In short, in the parasite life cycle sporozoites are delivered by the bite of the infected mosquito, find their way to the liver, and invade hepatocytes. Two proteins have been identified as implicated in the recognition and invasion of the hepatocytes, CS (circumsporozoite) protein (Frevert, U., P. Sinnis, C. Cerami, W. Shreffler, B. Takacs, and V. Nussenzweig. 1993. Malaria circumsporozoite protein binds to heparan sulfate proteoglycans associated with the surface membrane of hepatocytes. J. Exp. Med. 177:1287-1298) and Thrombospondin-related anonymous protein or TRAP (Sultan, A. A., V. Thathy, U. Frevert, K. J. Robson, A. Crisanti, V. Nussenzweig, R. S. Nussenzweig, and R. Ménard. 1997. TRAP is necessary for gliding motility and infectivity of Plasmodium sporozoites. Cell 90:511-522). Antibodies to proteins on the parasite surface might conceivably neutralize sporozoites and prevent subsequent development of liver stages. In the hepatocyte the parasite differentiates and replicates asexually as a schizont to produce enormous amounts of merozoites that will initiate the infection of red blood cells. Antigens specific for the liver stage have been identified (Calle J M, Nardin E H, Clavijo P, Boudin C, Stuber D, Takacs B, Nussenzweig R S & Cochrane A H. 1992. Recognition of different domains of the Plasmodium falciparum CS protein by the sera of naturally infected individuals compared with those of sporozoite-immunized volunteers.J Immunol. 49(8):2695-701; Nardin, E H & Nussenzweig, R S.1993.T cell responses to pre-erythrocytic stages of malaria: role in protection and vaccine development against pre-erythrocytic stages.Annu Rev Immunol. 1993;11:687-727).

It has been proposed that these antigens together with those from the sporozoites are in part processed by the host cell and presented on the surface together with class I MHC molecules. This presentation can lead to the recognition by cytotoxic T-lymphocytes and killing of the infected cells or stimulation of the T cells to produce cytokines can ultimately lead to the death of the intracellular parasite.

Merozoites surviving the pre-erythrocytic stages initiate the asexual blood stage infection, which is responsible for the disease. The parasite infects erythrocytes which do not express class I molecules and therefore cytotoxic T-lymphocytes are not important. Antibody binding to the surface of the merozoite probably plays a major role in immunity to asexual blood stages. Potentially these antibodies could neutralize parasites or lead to Fc-dependent mechanisms of parasite killing, e.g., macrophages. Complete protection against sporozoite challenge observed in irradiated P. berghei sporozoite-immunized mice and P.falciparum sporozoite-inmunized humans results from immune responses to antigens expressed by the parasite at the preerythrocytic stages of its life cycle (Nardin E H, Nussenzweig R S 1993. T cell responses to pre-erythrocytic stages of malaria: role in protection and vaccine development against pre-erythocytic stages. Ann. Rev. Immunol. 11, 687-). Antibody, CD4⁺ and CD8⁺ T cells have been implicated in preerythrocytic immunity (Schofield L, Villaquiran J, Ferreira A, Schellekens H, Nussenzweig R, Nussenzweig V 1987. γ-interferon, CD8⁺ T cells and antibodies required for immunity to malaria sporozoites Nature 330: 664-666; Rodrigues M, Li S, Murata K, Rodriguez D, Rodriguez J R, Bacik I, Bennink J R, Yewdell J W, Garcia-Sastre A, Nussenzweig R S, Esteban M, Palese P, Zavala F 1994. Influenza and vaccinia viruses expressing malaria CD8⁺ T and B cell epitopes. Comparison of their immunogenicity and capacity to induce protective response. J.Immunol. 153, 4636-4648).

There are 6 main preeythrocytic antigens in P.falciparum parasites: the circunsporozoite protein (CS), thrombospondin related adhesion protein (TRAP), liver-stage antigens 1 and 3 (LSA-1 and 3), Pfs 16 and sporozoite threonine and asparagine-rich protein. A number of epitopes identified on the different plasmodial proteins are being expressed in different systems towards immunogenicity studies (Munesinghe D Y, Clavijo P, Calle M C, Nardin E H, Nussenzweig R S 1991. Immunogenicity of multiple antigen peptides (MAP) containing T and B cell epitopes of the repeat region of the P.falciparum circunsporozoite protein. Eur.J.Immunol. 21, 3015-3020; Rodrigues et al 1994; Shi Y A, Hasnain S E, Sacci J B, Holloway B P, Fujioka H, Kumar N, Wohlhueter R, Hoffman S L, Collins W E, Lal A A 1999. Immunogenicity and in vitro efficacy of a recombinant multistage Plasmodium falciparum candidate vaccine. Proc. Natl. Acad. Sci. USA 96, 1615-1620; Aidoo M, Lalvani A, Gilbert S C, Hu J T, Daubersies P, Hurt N, Whittle H C, Druilhe P, Hill A V S 2000. Cytotoxic T-lymphocyte epitopes for HLA-B53 and other HLA types in the malaria vaccine candidate liver-stage antigen 3. Infect.Immun. 68, 227-232).

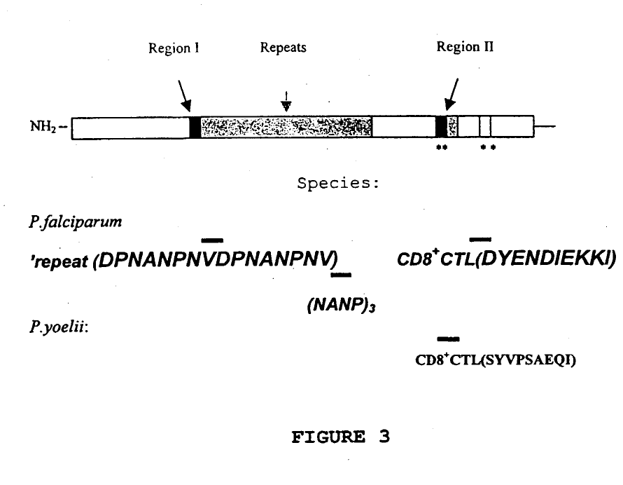

FIG. 3 shows a schematic representation of the CS protein of Plasmodium sp. (Nardin e Nussenzweig, 1993) and the location of epitopes expressed by recombinant YF 17D viruses of the present invention. The CS protein contains an immunodominant B epitope located in its central area This epitope consists of tandem repeats of species-specific amino acid sequences. In P.falciparum this epitope, asparagine-alanine-asparagine-proline, (NANP) has been detected in all isolates and thus represents an ideal target for vaccine development. Initial clinical trials with synthetic or recombinant peptides administered with alum resulted in induction of rather modest levels of antibodies to sporozoites and only individuals with the highest levels of antibodies were protected against P.falciparum with delayed onset of parasitemia in others (Hoffman S L, Nussenzweig V, Sadoff J C, Nussenzweig R S 1991. Progress toward malaria preerythrocytic vaccines. Science 252: 520-521). Other studies in animals of passive transfer of monoclonal antibodies to the repeats of the CS protein also suggest that antibodies can provide protection against sporozoite infection. The problem that remains is to engineer vaccines that elicit levels of antibodies in humans with the appropriate specificity and affinity to destroy all of the sporozoites before they enter the hepatocytes. In this regard it is noteworthy that immunization of humans with a single dose YF 17D virus suffices to induce detectable levels of neutralizing antibodies to YF even after 40 years of the primary immunization (Monath, 1999) and that lends further support for the applicant of the present invention to use 17D virus to express relevant pathogens epitopes, especially the relevant malarial epitopes, towards the development of new live viral vaccines capable of protecting, being life-long against yellow fever and a second disease, for example malaria. The liver stage of the parasite is also target for vaccine development because it offers additional antigens, and in contrast to the short-lived existence of sporozoites in the bloodstream of the mammal host, human malarias develop in the liver for several days. The effector mechanisms against these intrahepatocytic forms are probably cytotoxic T cells that destroy the infected hepatocytes and γ-interferon that inhibits parasite development.

Preerythrocytic immunity to Plasmodium is mediated in part by T lymphocytes acting against the liver stage parasite. These T cells must recognize parasite-derived peptides on infected host cells in the context of major histocompatibility complex antigens. T-cell-mediated immunity appears to target several parasite antigens expressed during the sporozoite and liver stages of the infection. A number of such CTL epitopes, present on different proteins of the preerythrocytic stages, have been identified in humans living in malaria endemic areas and are restricted by a variety of HLA class I molecules (Aidoo M, Udhayakumar V 2000 Field studies of cytotoxic T lymphocytes in malaria infections: implications for malaria vaccine development. Parasitol. Today 16, 50-56).

Cytotoxic T cells, mostly CD8⁺, which require the class I antigen presentation pathway are primarily generated by intracellular microbial infections, and have been most thoroughly investigated in viral infections. Recombinant viruses expressing the desired foreign epitopes, are therefore a logical approach towards generating the cytotoxic T cells of the desired specificity.

Miyahira et al (Miyahira Y, Garcia-Sastre A, Rodriguez D, Rodriguez J R, Murata K, Tsuji M, Palese P, Esteban M, Zavala F, Nussenzweig R S 1998. Recombinant viruses expressing a human malaria antigen can elicit potentially protective immune CD8⁺ responses in mice. Proc. Natl. Acad. Sci. USA 95, 3954-3959) have studied in a mouse model the immunogenicity of a CTL epitope located on CS of P.falciparum. The CTL epitope (DELDYENDIEKKICKMEKCS) was expressed in a bicistronic neuraminidase gene of the influenza D strain. Recombinant vaccinia included the whole CS gene containing both humoral and CTL epitopes. Immunization of mice with either flu or vaccinia elicited a modest CS-specific CD8⁺ T cell response detected by interferon γ secretion of individual immune cells. Priming of mice with the recombinant flu virus and boosting with the vaccinia recombinant resulted in a striking enhancement of this response.

A vaccinia virus expressing several P.falciparum antigens was developed and used in a clinical trial. While cellular immune responses were elicited in over 90% of the individuals antibody responses were generally poor. Of the 35 volunteers challenged, only one was completely protected, although there was a significant delay on the onset of parasitemia (parasitemia (Ockenhouse C F, Sun P F, Lanar D E, Wellde B T, Hall B T, Kester K, Stoute J A, Magill A, Krzych U, Farley L, Wirtz R A, Sadoff J C, Kaslow D C, Kumar S, Church L W, Crutcher J M, Wizel B, Hoffman S, Lalvani A, Hill A V, Tine J A, Guito K P, de Taisne C, Anders R, Ballou W R, et al.. 1998. Phase I/IIa safety, immunogenicity, and efficacy trial of NYVAC-Pf7, a pox-vectored, multiantigen, multistage vaccine candidate for Plasmodium falciparum malaria. J Infect Dis. 177: 1664-1673).

Recombinant Sindbis virus expressing a minigene encoding the CD8⁺ T-cell epitope SYVPSAEQI of the CS protein of rodent malaria parasite P.yoelii when inoculated subcutaneously in mice induced a large epitope-specific CD8⁺ T-cell response. This immunization also protected mice against infection by sporozoites (Tsuji M, Bergmann C C, Takita-Sonoda Y, Murata K, Rodrigues E G, Nussenzweig R S, Zavala F 1998. Recombinant Sindbis virus expressing a cytotoxic T-lymphocyte epitope of a malaria parasite or of influenza virus elicit protection against the corresponding pathogen in mice. J. Virol. 72, 6907-6910).

It has also been shown that mice immunized by a single dosis of a recombinant adenovirus expressing the CS protein of P.yoelii elicits a high degree of resistance to infection mediated primarily by CD8⁺ T cells (Rodrigues E G, Zavala F, Eichinger D, Wilson J M, Tsuji M 1997. Single immunizing doses of recombinant adenovirus efficiently induces CD8⁺ T cell-mediated protective immunity against malaria. J. Immunol. 158, 1268-1274).

Because of the complexity of the parasite and its life cycle there is a consensus that a highly effective malaria vaccine would require a combination of key antigens and/or epitopes from different stages of the life cycle and that induction of both humoral and cellular immunity is required for optimal efficacy. Such a vaccine would also circumvent the problems with host genetic restriction and antigenic variability in the case of single antigen-based vaccines. So far, however, several attempts to attain protective immunity with different antigens preparations from several stages have not produced convincing results.

The critical issues for the multivalent approach as with single antigen are the identification of antigens that will induce a (partially) protective response in all or most of the target population, the delivery of these antigens in a form that will stimulate the appropriate response and the delivery system must allow presentation of the antigens in a form that stimulates the immune system. The development described here which utilizes flaviviruses for the expression of defined pathogen antigens/epitopes should address the issues of presentation to the target population. Regarding the YF 17D virus, it is an extremely immunogenic virus, inducing high antibody seroconversion rates in vaccinees of different genetic background.

The applicant of the present invention particularly explores the feasibility of using the YF 17D virus strain and substrains thereof, not only as a very effective proven yellow fever vaccine, but also as a vector for protective antigens, particularly protective epitopes. This will result in the development of a vaccine simultaneously effective against yellow fever and other diseases which may occur in the same geographical areas such as malaria, dengue, Japanese encephalitis, tick-borne encephalitis, fungi infections, etc.

The main goal was to establish a general approach to insert and express single defined antigens, including epitopes into sites structurally apart from areas known to interfere with the overall flavivirus E protein structure, specially into the fg loop or the E₀F₀ loop of the E protein of a given flavivirus, such as yellow fever, dengue, Japanese encephalitis, tick-borne encephalitis, that can be used as new live vaccine inducing a long lasting and protective immune response. More particularly, the present invention is related to a general approach to express single defined epitope on the fg loop of the E protein of a YF 17D virus.

As used herein, the term “Flavivirus” means wild virus, attenuated virus and recombinant virus, including chimeric virus.

The genetic manipulation of the YF 17D genome was carried out by using the YF infectious cDNA as originally developed by Rice et al (1989) which consists of two plasmids named pYF5′3′IV and pYFM5.2. The YF genome was splited in two plasmids due to the lack of stability of some virus sequences in the high copy number plasmid vector, pBR322. However, in the case of dengue 2 virus, full length cDNA was steady cloned in the same plasmid (Kinney R M, Butrapet S, Chang G J, Tsuchiya K R, Roehrig J T, Bhamarapravati N & Gubler D J. 1997. Construction of infectious cDNA clones for dengue 2 virus: strain 16681 and its attenuated vaccine derivative, strain PDK-53. Virology. 230 :300-308).When cut by specific restriction enzymes (Apal or NsiI and AatII) specific restriction fragments are generated which upon ligation reconstitute the complete YF genome in the cDNA form. Restriction of this cDNA form with XhoI linearizes the cDNA and allows in vitro synthesis of capped RNA from the SP6 promoter sequence. Such RNA when transfected into cultured cells gives rise to infectious virus. The phenotype of this virus has been tested in monkeys (Marchevsky R S, Mariano J, Ferreira V S, Almeida E, Cerqueira M J, Carvalho R, Pissurno J W, Travassos da Rosa A P A, Simões M C, Santos C N D, Ferreira I I, Muylaert I R, Mann G F, Rice C M and Galler R. 1995. Phenotypic analysis of yellow fever virus derived from complementary DNA. Am. J. Trop. Med. Hyg. 52, 75-80) and these studies indicated the need for genetic modification of the cDNA as to make the resulting virus more attenuated. This has been carried out by Galler and Freire (U.S. Pat. No. 6,171,854) and resulted in new versions of pYF5′3′IV and pYFM5.2, named G1/2 and T3/27, respectively. The physical map and sequences of both plasmids are shown elesewhere and these plasmids have been deposited at ATCC (ATCC #97771 and ATCC #97772).

Having the genome split into two plasmids is convenient for insertion cloning purposes but not ideal for the recovery of the virus. So, in order to accomplish a higher specific infectivity of the transcripts the applicant developed a full-length cDNA clone for the YF genome. It was identified in the literature plasmid pACNR1180 (Ruggli N; Tratschin J-D.; Mittelholzer C; Hofmann M A. 1996. Nucleotide sequence of classical swine fever virus strain Alfort/187 and Transcription of infectious RNA from stably cloned full-length cDNA. J. Virol. 70, 3478-3487) which has a replication origin named P15A that allows only limited replication of the plasmid reducing the number of plasmid DNA molecules per bacterial cell. This plasmid system was used by the authors to stabilize the genome of the classical swine fever virus with a genome size close to that of YF virus. This plasmid, pACNR1180 was provided by Dr. J D Tratschin of Institute of Virology and Immunoprophylaxis, Switzerland.

Besides pACNR1180, other vectors which provide the stabilization of the YF virus genome can be used to prepare the plasmids of the present invention. Specific examples include plasmids which have a replication origin that allows only limited replication of the plasmid reducing the number of plasmid DNA molecules per bacterial cells, i.e. vectors consisting of low copy number plasmids such as pBeloBAC11 (Almazan F, Gonzalez J M, Penzes Z, Izeta A, Calvo E, Plana-Duran J, Enjuanes L. 2000 Engineering the largest RNA virus genome as an infectious bacterial artificial chromosome.ProcNatlAcadSciUSA97:5516-5521). Another possibility is the use of high copy number plasmids such as pBR322.

The new version of pACNR1180 was named pACNR1180Nde/Sal. It was obtained by removing most of the unique restriction sites of pACNR1180 by digestion with NdeI/SalI, filling in the ends by treating with Klenow enzyme, ligating and transforming E.coli XL1-blue.

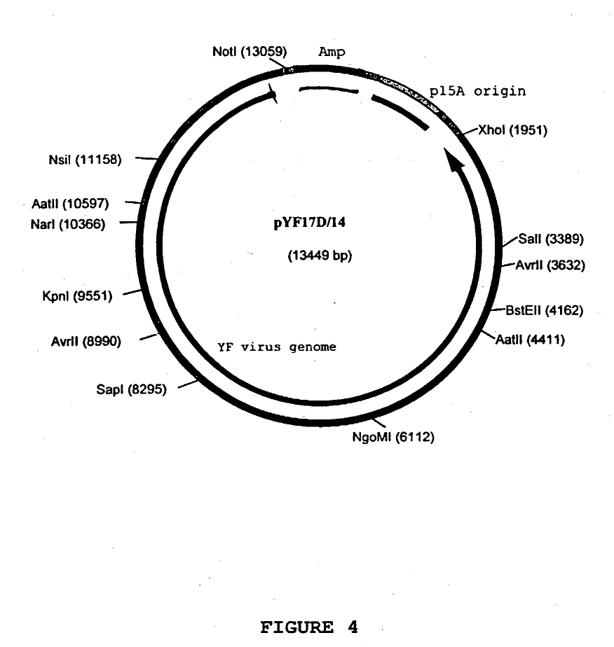

To generate pYF17D/14, fragments NotI (13,059)-NsiI (11,158) and SalI (3,389)-XhoI (1951) from G1/2 plasmid (described in detail in U.S. Pat. No. 6,171,854) were first ligated to NotI/XhoI-digested pACNR1180Nde/Sal. The resulting plasmid was named NSK7 (map not shown) and used to clone the remaining of the YF cDNA contained in plasmid T3/27 (described in detail in U.S. Pat. No. 6,171,854) by digestion with NsiI (11,158)-SalI (3,389), ligation of the appropriate fragments and transformation of the same bacterium. Restriction enzyme mapping confirmed the expected physical structure of a plasmid containing the complete YF cDNA. A total of 8 clones were analysed, virus was recovered from 5 out of 6 tested and complete nucleotide sequence determination confirmed the expected YF sequence. The plasmid contains 13449 base pairs and was named pYF17D/14 (FIG. 4).

pYF17D/14 contains an ampicillin resistance gene from position 13,196 to 545 and the p15A origin of replication (nts 763 to 1585) both derived from plasmid pACYC177 (Ruggli et al, 1996). Nucleotides 12,385 to 12,818 correspond to the SP6 promoter. The YF genome is transcribed in this plasmid from the opposite strand as the complete genome spans nucleotide 12,817 to 1951. All insertions at the fg loop of the yellow fever virus E protein are made at the EcoRV site of YFE200 plasmid and from there incorporated into pYF17D/14 by exchanging fragments NsiI/NotI. Other representative sites are shown in FIG. 4.

Plasmid G1/2 contains the YF 5′ terminal sequence (nt 1-2271) adjacent to the SP6 phage polymerase promoter and 3′ terminal sequence (nt 8276-10862) adjacent to the XhoI site used for production of run off transcripts. To allow the insertion of foreign sequences into the fg loop as defined by the analysis of the 3D structure of the E protein we have created by in vitro mutagenesis a restriction site (EcoRV) at nucleotide 1568. The creation of this site led to two amino acid changes in the E protein at positions E-199 and E-200 (E→D, T→I, respectively).

It is important to emphasize that the creation of the restriction site of choice is dependent on the nucleotide sequence that makes up each loop and will vary according to the Flavivirus genome sequence to be used as vector. Therefore, the restriction site used for one Flavivirus EcoRV site is specific to the fg loop of yellow fever but (cortar?) may not be useful for insertion into the genome of other flavivirus. Those skilled in the art will identify suitable sites by using conventional nucleotide sequence analysis software for the design of other appropriate restriction sites.

It is noteworthy that amino acid 200 is a K in Asibi, T in all 17D viruses analyzed and I in E200. The fact that E200 is a position that is altered in all 17D viruses would suggest that particular alteration is important for the attenuation of 17D virus and alterations there might compromise that trait. However, the mutation introduced for the creation of the insertion site does not lead to reversion to the original amino acid and both are very distinct in character. Moreover, it is likely that attenuation of 17D is multifactorial, and not only related to the structural region as suggested the phenotype of chimeric 17D/JE-Nakayama in the mouse model of encephalitis (Chambers et al, 1999). In addition no major alterations in the E protein structure was apparent when modelling these amino acids changes into the predicted 3D model. Finally, the YF17D/G1/2-derived virus grew in tissue culture to the same levels and was as neurovirulent for mice as the parental 204/DD virus derived from G1/2/T3/27 cDNA.

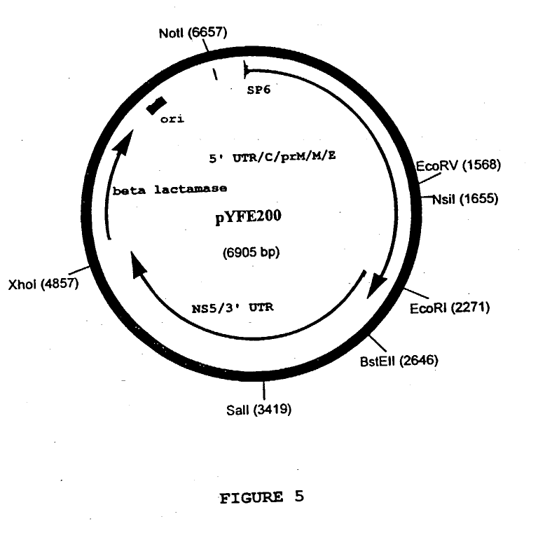

In order to create the EcoRV site in the YF sequence we subcloned a NotI/ApaI fragment of G1/2 into the pAlter plasmid (Promega Inc) and performed the mutagenesis as indicated by the manufacturer in the presence of mutagenizing synthetic oligonucleotide specifying the desired EcoRV site. Mutants bearing the site were identified by restriction enzyme analysis of plasmid DNA and the ApaI/NotI fragment contained in one of the clones was sequenced entirely to confirm the restriction site was the sole mutation in the YF sequence. Such fragment was cloned back into G1/2 plasmid and the resulting plasmid was named pYFE200, the map of which is shown in FIG. 5.

This plasmid was derived from pYF5′3′IV originally described by Rice et al, 1989 as modified by Galler and Freire (U.S. Pat. No. 6,171,854) and herein. It contains 6905 nucleotides and region 1-2271 corresponds to the 5′ UTR, C, prM/M and E genes. This region is fused through an EcoRI site at the E gene (2271) to another EcoRI site in the NS5 gene (position 8276). At position 1568 in the E gene we created the EcoRV site which is used for epitope insertion into the E protein fg loop. This plasmid also consists of the NS5 gene from nucleotide 8276 to the last YF genome nucleotide (10,862) containing therefore part of the NS5 gene and the 3′ UTR. Nucleotides 5022 to 5879 correspond to the ampicillin-resistance gene and 6086 to 6206 to the origin of replication, both derived from pBR322 plasmid. Besides pBR322, other vectors known to specialists in the art may be used such as pBR325, BR327, pBR328,pUC7, pUC8, pUC9, pUC19, λ phage, M13 phage, etc.. The location of relevant restriction enzyme sites is shown in FIG. 5.

YFE200 plasmid has been deposited at ATCC under number PTA2856. pYFE200 was used to produce templates together with T3/27 which allowed the recovery of YF virus that resembles YFiv5.2/DD virus (U.S. Pat. No. 6,171,854) in growth properties in Vero and CEF cells, plaque size, protein synthesis and neurovirulence for mice (data for E200 and the recombinants derived thereof are shown in the examples).

The template to be used for the regeneration of YF 17D virus is prepared by digesting the plasmid DNA (YFE200 and T3/27) with NsiI and SalI. After digestion with Xhol to linearize the ligated DNA, the template was used for in vitro transcription. Virus has been recovered after RNA transfection of cultured animal cells.

The animal cell culture used herein may be any cell insofar as YF virus 17D strain can replicate. Specific examples include, Hela (derived from carcinoma of human uterine cervix), CV-1 (derived from monkey kidney), BSC-1 (derived from monkey kidney),RK 13 (derived from rabbit kidney), L929 (derived from mouse connective tissue), CE (chicken embryo) cell, CEF (chicken embryo fibroblast), SW-13 (derived from human adrenocortical carcinoma), BHK-21 (baby hammster kidney), Vero (african green monkey kidney), LLC-MK2 (derived from Rhesus monkey kidney), etc.

In a preferred embodiment of the present invention, Vero cells are the preferred substrate in all production steps as the titers obtained in different growth curves, as well as the genetic stability gave better results. Primary cultures of chicken embryo fibroblasts (CEF) may be a second choice to be used as substrate in all production steps as these cells have been used for measles vaccine production for years with extensive experience in its preparation and quality controls; a number of Standard Operating Practices (SOPs) is available and a patent application dealing with the production of YF vaccine in CEF cultures has been filled (EP 99915384.4)

Therefore, the flavivirus system described here, more particularly, the YF system, provides a powerful methodology for the development of unlimited formulations of recombinant viruses expressing different epitopes. It is anticipated that the appropriate formulation of several recombinant viruses should elicit the adequate immune response to cope with the different parasite stages.

The following examples are illustrative of the invention and represent preferred embodiments. Those skilled in the art may know, or be able to find using no more than routine experimentation, to employ other appropriate materials and techniques, such as the above mentioned vectors, culture cells and transfection methods.

### EXAMPLE 1

Structural Analysis of the Insertion of Specific Protein Epitopes

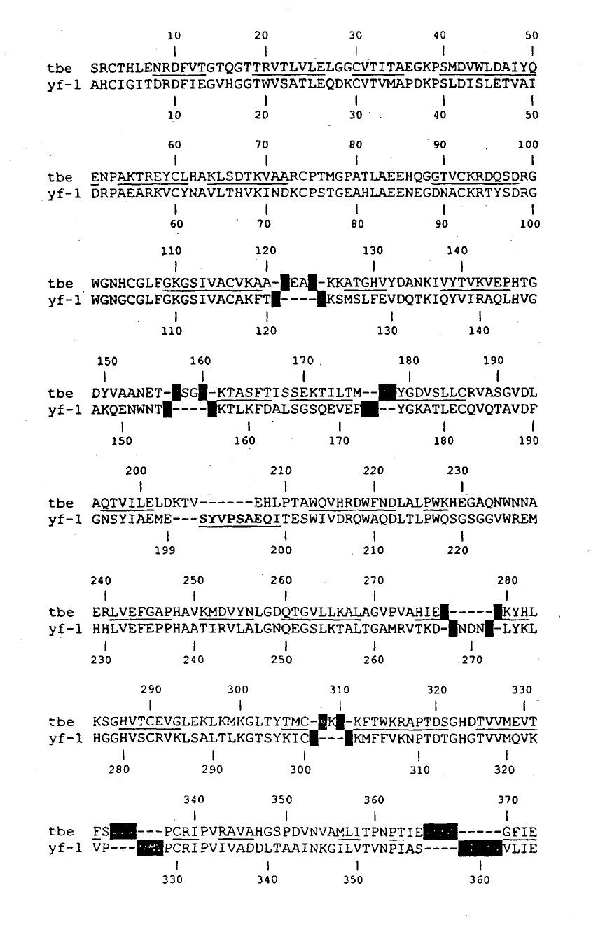

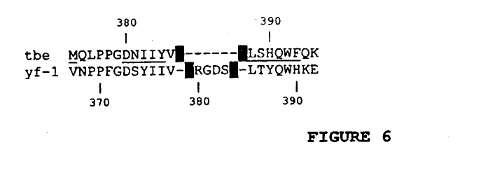

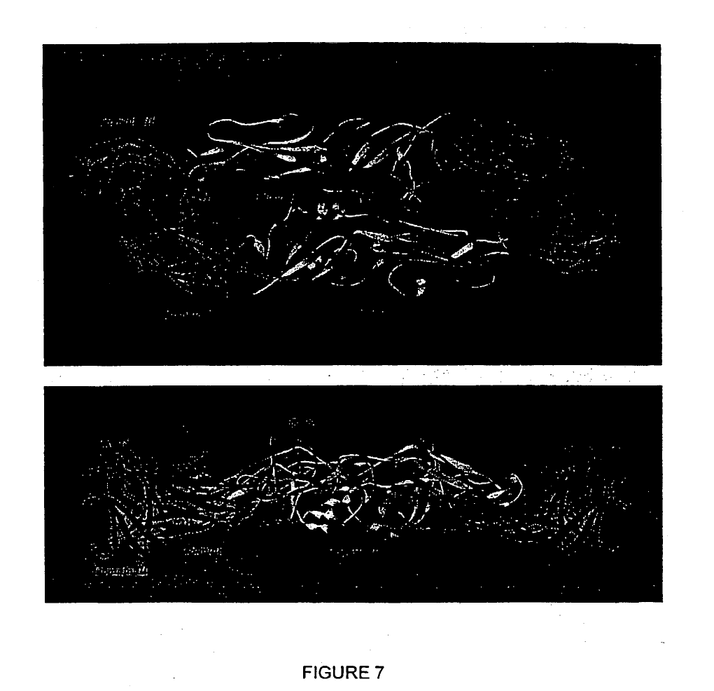

Ten models were produced for the insertion SYVPSAEQI in the fg loop region using the alignment shown in FIG. 6 in which the insertion is made between E199 and T200. The inserted residues will be referred to as 199A to 199I. The pseudo-energies of the best five models were comparable to those of the native yf model. Their structures are variable as one skilled in the art would expect from an insertion of nine residues in length. The variation in structure of the loop leads to correlated variation in the neighbouring loop between β-strands k and l. The glutamine sidechain of residue Gln199H (ie the eigth residue of the inserted peptide) in several of the best models shows a conformation compatible with the formation of a hydrogen bond via its N_{ε2} to the carbonyl of Val244 of the opposite monomer in a similar fashion to that made by the N_{δ1} of His208 in tbe. One representative model had an overall G-factor of 0.07, equivalent to a structure of <1.0 Å resolution and has good stereochemistry in the region of the insertion. The total Verify_{—}3D score for the segment from 199 to 200 (including the nine inserted residues) is +3.69 (a mean value of 0.34 per residue) indicating that the residues of the loop have been built into favourable chemical environments. By comparison the mean value per residue for the equivalent loop in the crystal structure of tbe is only 0.28. The average contact area per monomer for this model is 1.460 Å², comparable to that observed in the crystal structure of tbe, as anticipated by the introduction of the insertion close to the proximal interface (FIG. 7).

During the construction of the insertion site two substitutions were made to the amino acid sequence: E199D and T200I. The consequence of such substitutions was analyzed with reference to the model. The substitution E199D is not expected to have serious consequences as it is conservative in nature, is observed in tbe and may lead to a salt-bridge with K123. There is also no significant change in the quality index for this residue as determined by WHATIF. The substitution T200I appears acceptable as the insertion leads to a rotation of the T200 sidechain in many of the ten models resulting in it being directed towards a hydrophobic pocket close to W203, the aliphatic region of R263 and L245. The substitution also retains the ramification on Cβ.

Using an identical approach to that described above the insertion DYENDIEKKI was introduced into the yf E protein. An identical alignment to that shown in FIG. 6 was used in this case with the exception of the insertion sequence itself. Models were produced using an identical protocol to that described above for the SYVPSAEQI insertion and were of similar quality. Typically the models present PROCHECK G-factors of −0.1 (equivalent to crystal structures with a resolution between 1.0 and 1.5 Å) and have around 90% of the residues within the most favourable regions of the Ramachandran plot. Although the loop conformations obtained show considerable variation, several suggest the possible appearance of new inter-subunit interactions which may affect dimer stability and/or influence the pH dependent dimer to trimer transition. Potential salt-bridges suggested by the models include those between Glu199C, Asp199E and/or Glu199G (the third, fifth and seventh residues of the insertion respectively) with Arg243 (native yf numbering) of the opposite subunit as well as Lys199H with the carbonyl of Leu65 of the opposite subunit. The salt bridge seen in the native yf model between Arg263 of one monomer and Glu235 of the other, is retained. In none of the models did Lys199H form a hydrogen bond equivalent to that made by His208 to the opposite subunit in tbe, but a potential hydrogen bond to the carbonyl of Leu65 is possible. Lys199I may form a salt-bridge with Glu199 of the same subunit and such an interaction should be feasible even after the glutamic acid to aspartic acid substitution. On dimerization each subunit loses an average of 1,483 Å² of accessible surface area (based on one such model), comparable to that of tbe, principally due to the reinsertion of a large loop between β-strands f and g.

Of note is the fact that neither of the two loop insertions described lead to steric clashes between the subunits, they preserve good steochemistry as well as (in the case of the best models) yielding reasonable chemical environments for the amino acid sidechains of the insertion. This is achieved without an exaggerated increase in the contact area between monomers suggesting that there is indeed no spatial restriction to the proposed insertions.

Compared with the loop observed experimentally in the crystal structure of the E protein from the the virus, the two insertions described above are three residues longer. These additional residues are accommodated in most models by an additional protrusion of the structure on the external surface (although in one model a short stretch of α-helix is observed). This suggests that it may be possible to accommodate even larger peptides within this insertion site.

Ten models were generated for the insertion GG(NANP)₃GG and a protocol identical to that described in the previous two examples. Different from the previous two examples however is the fact that this insertion is a B-cell epitope, is considerably larger, includes a repeated tetrapeptide and is rich in proline residues. In this case in order that the B-cell epitope should be as exposed as possible (in order to be antibody accessible) and given adequate conformational freedom, as well as in facilitating its insertion into the fg loop in the form of an Ω loop (Leszczynski J F, Rose G D, 1986. Loops In Globular-Proteins—A Novel Category Of Secondary Structure Science 234, 849-855) two glycine residues (spacers) were added to both the N- and C-termini of the epitope, (NANP)₃. Due to the reduced size of the glycine sidechain and to its achiral Cα atom, a greater region of Ramachandran space is accessible to its mainchain dihedral angles, φ and ψ, increasing the chances of a sucessful insertion which does not prejudize the local structure in the region of the insert. This approach produced models which presented excellent stereochemistry (PROCHECK G-factors of the order of −0.1, equivalent to crystal structures of resolution 1.0 to 1.5 Å). The loop insertion itself is also free of stereochemical strain. We surmise that this is the result of the N- and C-terminal glycine spacers which serve to lift the loop free of the external surface of the protein. In several of the models one or more of these glycines adopt backbone conformations which would be prohibited for other amino acids. The remainder are generally in extended (β) conformations. These factors appear to emphasize the importance of their inclusion.

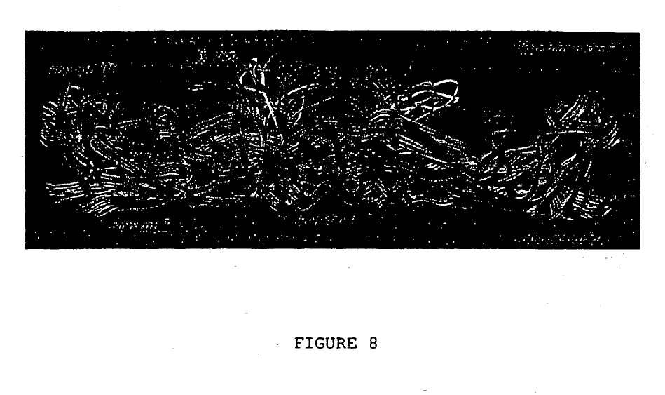

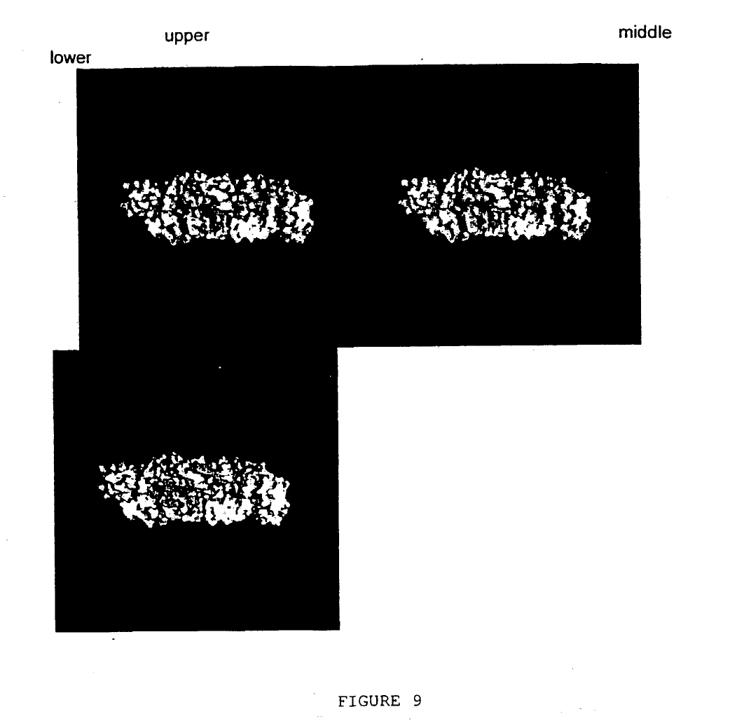

Those skilled in the art will know that a reliable prediction of the structure of such a loop is beyond current theoretical approaches. There is therefore considerable spread in the resulting conformation in the ten models generated (FIG. 8). This wide distribution of conformations (of approximately equal pseudo-energy) does not lead to significant alteration of the remainder of the structure, emphasizing that the loop has access to a large volume of space, as originally designed. This does not mean that the loop would necessarily be unstructured, merely that many possible structures are accessible to the loop, increasing the probability that that which is immunologically relevant may be adopted, even within the context of its insertion into the yf E protein. FIG. 9 illustrates better the volume considerations.

The (NANP)₃ sequence in the ten models has a mean relative accessible surface area (compared to its unfolded structure) of 63.7%. This compares with a mean value of 27.4% for the structure overall, demonstrating that the insertion has a very large relative accessibility, as intended. If the glycine spacers are eliminated this value for the (NANP)₃ sequence falls to 53.6%, demonstrating that the spacers have a role in increasing the exposure of the epitope. Examination of the models shows that increasing the length of the glycine spacer beyond two residues would appear to bring no additional advantage in exposing the epitope but may represent an entropic cost for the structure which could lead to its destabilization. Two glycines appears the optimum to us.

It is a reasonable expectation that the envelope proteins of japanese encephalitis virus and Dengue 2 would accommodate the above described insertions equally well as yellow fever virus. The alignment of FIG. 2 shows that a similar six-residue deletion is present in all three viral envelope proteins compared to tbe. Models for the je and d2 E protein, produced using a similar protocol to that for yf, frequently show a β-turn structure for the fg loop, stabilized by a mainchain hydrogen bond. A similar β-turn is also observed in yf if the alignment restraints are relaxed around residues of the fg loop itself. The models for je show a potential intersubunit salt bridge between Lys201 with Glu243 (je numbering) of the opposite subunit. This glutamic acid in yf interacts with Arg263 which has been substituted by valine in je. Similar contacts to those of yf are also observed around the distal dimer interface site. A representative model for the je E protein has a PROCHECK G-factor of −0.1, 89.9% of residues in the most favourable regions of the Ramachandran plot, good sterochemistry in the region of the fg loop (which adopts a type I β-turn), a good WHATIF quality score for the fg loop (residues 203 to 212 yielding and average of 0.768) and buries a mean accessible surface area of 1,048 Å² per subunit on dimerization. Similar results are obtained for d2, in which the fg loop adopts either the type I or type II β-turn conformation. From these data those skilled in the art will be able to apply the insertion strategy described above for yf to other flaviviruses such as je and d2.

The site which comprises the region of β-strands f and g including the fg loop which form part of the five-stranded anti-parallel β-sheet of domain II of the flavivirus envelope protein comprises the region of amino acid 196 to 215 with reference to the tick-borne encephalitis virus sequence described in FIG. 2. More particularly, the site is the loop area between β-strands f and g which form part of the five-stranded anti-parallel β-sheet of domain II of the flavivirus envelope protein (amino acid 205 to 210 with reference to the tick-borne encephalitis virus sequence described in FIG. 2).

Additionally, the site which comprises the region of E₀ and F₀ strands including the E₀F₀ loop which form part of the eight stranded β-barrel of domain I of the flavivirus envelope protein comprises the region of amino acid 138 to 166 with reference to the tick-borne encephalitis virus sequence described in FIG. 2. More particularly, the site is the loop area between E₀ and F₀ strands which form part of the eight stranded β-barrel of domain I (amino acid 146 to 160 with reference to the tick-borne encephalitis virus sequence described in FIG. 2).

### EXAMPLE 2

a) Derivation of Plasmid pACNR1180Nde/Sal.

pACNR1180Nde/Sal, the new version of plasmid pACNR1180, is obtained by removing most of the unique restriction sites of pACNR1180 by digestion with NdeI/SalI, filling in the ends by treating with Klenow enzyme, ligating and transforming E.coli XL1-blue. This new version of pACNR1180 was named pACNR1180Nde/Sal.

b) Derivation of Plasmid pYF17D/14

To generate pYF17D/14, fragments NotI (13,059)-NsiI (11,158) and SalI (3,389)-XhoI (1951) from G1/2 plasmid (described in detail in U.S. Pat. No. 6,171,854) were first ligated to NotI/XhoI-digested pACNR1180Nde/Sal. The resulting plasmid was named NSK7 (map not shown) and used to clone the remaining of the YF cDNA contained in plasmid T3/27 (described in detail in U.S. Pat. No. 6,171,854) by digestion with NsiI (11,158)-SalI (3,389), ligation of the appropriate fragments and transformation of the same bacterium. The plasmid contains 13449 base pairs and was named pYF17D/14 (FIG. 4).

pYF17D/14 contains an ampicillin resistance gene from position 13,196 to 545 and the p15A origin of replication (nts 763 to 1585) both derived from plasmid pACYC177 (Ruggli et al, 1996). Nucleotides 12,385 to 12,818 correspond to the SP6 promoter. The YF genome is transcribed in this plasmid from the opposite strand as the complete genome spans nucleotide 12,817 to 1951. All insertions at the fg loop of 17D virus E protein are made at the EcoRV site of YFE200 plasmid and from there incorporated into pYF17D/14 by exchanging fragments NsiI/NotI. Other representative sites are shown in FIG. 4.

c) Derivation of Plasmid YFE200.

In order to create the EcoRV site in the YF sequence we subcloned a NotI/ApaI fragment of G1/2 into the pAlter plasmid (Promega Inc) and performed the mutagenesis as indicated by the manufacturer in the presence of mutagenizing synthetic oligonucleotide specifying the desired EcoRV site. Mutants bearing the site were identified by restriction enzyme analysis of plasmid DNA and the ApaI/NotI fragment contained in one of the clones was sequenced entirely to confirm the restriction site was the sole mutation in the YF sequence. Such fragment was cloned back into G1/2 plasmid and the resulting plasmid was named YFE200, the map of which is shown in FIG. 5.

d) Preparation of Large Amounts of Plasmid DNA

To prepare plasmids DNAs from bacteria, glycerol stocks of the E. coli harboring each of the two YF plasmids, pYFE200 and pYF17D/14 must be available. Luria Broth-50% glycerol media is used in the preparation of the stocks, which are stored at −70° C. Frozen aliquots of the pDNA are also available.

The bacteria are grown in 5 ml LB containing ampicillin (50 μg/ml) for YFE200 and ampicillin (50 μg/ml) plus tetracyclin (15 μg/ml) overnight at 37° C. for NSK14-harboring bacteria. This is used to inoculate 1:100 large volumes of LB (usually 100-200 ml). At OD₆₀₀ of 0.8, chloramphenicol is added to 250 μg/ml for the amplification of the plasmid DNA and incubated further overnight. The plasmid is extracted using the alkaline lysis method. The final DNA precipitate is ressuspended in TE (Tris-EDTA buffer) and cesium chloride is added until a refraction index of 1.3890 is reached. The plasmid DNA is banded by ultracentrifugation for 24 hours. The banded DNA is recovered by puncturing the tube, extracting with butanol and extensive dialysis.

The yields are usually 1 mg of pDNA/liter of culture for pYFE200 and 0.02 mg/liter for pYF17D/14. pYFE200 was deposited on Dec. 21, 2000 under accesion number PTA-2856 with the American Type Culture Collection (ATCC), 10801 University Blvd., Manassas, Va. 20110-2209.

### EXAMPLE 3

Preparation of DNA Template:

The template to be used for the regeneration of YF 17D virus is prepared by digesting the plasmid DNA (YFE200 and T3/27) with NsiI and SalI (Promega Inc.) in the same buffer conditions, as recomended by the manufacturer. Ten μg of each plasmid are digested with both enzymes (the amount required is calculated in terms of the number of pmol-hits present in each pDNA in order to achieve complete digestion in 2 hours). The digestion is checked by removing an aliquot (200 ng) and running it on 0.8% agarose/TAE gels. When the digestion is complete, the restriction enzymes are inactivated by heating.

Linearization of the DNA resulting from the ligation of both NSiI/SalI-digested plasmids is carried out by the use of XhoI, and is performed with buffer conditions according to the manufacturer (Promega). The resulting product is thereafter phenol-chloroform extracted and ethanol precipitated. The precipitate is washed with 80% ethanol and resuspended in sterile RNase-free Tris-EDTA buffer. A template aliquot is taken for agarose gel analysis together with commercial markers for band sizing and quantitation. The template is stored at −20° C. until use for in vitro transcription.

### EXAMPLE 4

RNA Transcription from cDNA Template of the Present Invention:

Preparation of the DNA template from the full-length pYF17D/14 clone for in vitro transcription is simpler as it requires less pDNA, usually 1-2 μg which is digested with XhoI for linearization. Digestion, cleaning of the DNA and quality of the template are carried out as described above. Transcription is as described in U.S. Pat. No. 6,171,854. RNA produced from XhoI-linearized pYF17D/14 DNA templates were homogeneous and mostly full-lenght in contrast to the two-plasmid system-derived RNA (data not shown).

### EXAMPLE 5

RNA Transfection:

Transfection of such RNA were carried out in a similar manner as described in U.S. Pat. No. 6,171,854 (Galler and Freire). Transfection of such RNA gave rise to virus which was similar to YFiv5.2/DD in its plaque size, growth in Vero and CEF cells, neutralization by hyperimmune sera to YF and protein synthesis.

### EXAMPLE 6

Construction of the Recombinant YF17D/8 Virus Expressing a Humoral B Cell Epitope.

For the expression of the (NANP)₃ humoral epitope of P.falciparum (see Table 1 below) two surrounding glycines were added on each side as to compensate for the likely formation of β-turn in the epitope given the presence of consecutive proline and asparagines and give the loop a more flexible structure. Therefore, the synthetic oligonucleotide insertion at the EcoRV site of pYFE200 plasmid which corresponds to the amino acid sequence depicted in Table 1 gives rise to plasmid pYFE200/8. This plasmid was deposited on Dec. 21, 2000 under accesion number PTA-2857 with the American Type Culture Collection (ATCC), 10801 University Blvd., Manassas, Va. 20110-2209.

Blunt-ended ligation of the synthetic oligonucleotide disrupts the EcoRV site and that was used to screen for plasmids bearing the insertion. EcoRV⁻ plasmids were sequenced around the insertion region to verify the orientation of the insert. Plasmids with correct insert orientation which therefore kept the required open reading frame were used for further sequence encompassing the whole structural region contained in this plasmid, more specifically, the area comprised between the NotI and NsiI sites. The NotI/NsiI cDNA fragment of 1951 bp was ligated to the NsiI/MluI fragment of 1292 bp of the the T3 plasmid and the NotI/MluI backbone of 10,256 bp of the full lenght clone pYF17D/14. Resulting plasmids were first screened for size and thereafter for the production of infectious transcripts by lipid-mediated RNA transfection of cultured Vero cells as described (Galler and Freire, U.S. Pat. No. 6,171,854). The resulting virus was named 17D/8. After transfection, YF 17D/8 had a titer (measured by plaquing on Vero cell monolayers) of about 4.0 log₁₀ PFU/ml. After one-single passage in Vero cells viral stocks had a titer of 6.1 log₁₀ PFU/ml. The presence of the insert in the viral genome was checked by sequencing the cDNA made to the virus present in the cell culture supernatant derived from the transfection.

TABLE 1
Amino acid sequence and specificity of
(NANP)₃ humoral epitope for
insertion into YF E protein

| | Antigen | | |
|---|---|---|---|
| Sequence | epitope | source | Clone |
| EMD GGNANPNANPNANPGG IES | CSP-B | P.falciparum | 17D/8 |

### EXAMPLE 7

Epitope Expression by the YF 17D/8 Recombinant Virus

To investigate the expression of the 16-amino acid epitope in the E protein of 17D/8 virus it was performed an indirect immunofluorescence assay using a monoclonal antibody directed to the (NANP)₃ repeat ((MAb 2A10; Zavala F, Cochrane A H, Nardin E H, Nussenzweig R S, Nussenzweig V 1983. Circunsporozoite proteins of malaria parasites contain a single immunodominant region with two or more identical epitopes. J.Exp.Med. 157, 1947-1957) and a mouse polyclonal hyperimmune serum to YF 17D (ATCC). The IFA was made using glutaraldehyde-fixed VERO cells infected for 48 h with YF17D/14 virus or with recombinant virus YF17D/8 carrying (NANP)₃ epitope at moi (multiplicity of infection) of 1. The samples were treated with twofold dilutions of YF-Hiperimmune ascitic fluid (ATCC) and mouse IgG directed against the immeunodominant B cell epitope NANP of P.falciparum CS protein purified from 2A10 monoclonal antibody as described (Zavala et al, 1983, a gift of Dr. M. Rodrigues, Escola Paulista de Medicina). Positive cells were evidenced by the binding of FITC-conjugated anti-mouse IgG.

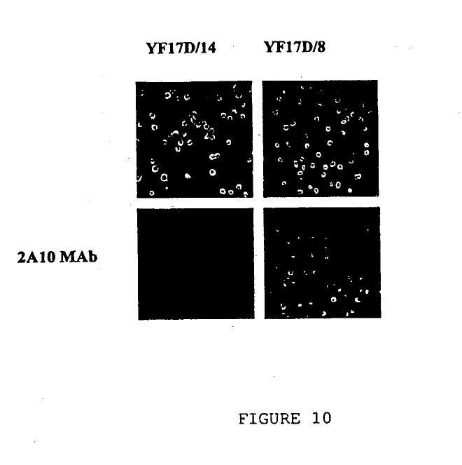

Only Vero cells infected with 17D/8 virus were recognized by mab 2A10 whereas the YF hyperimmune serum detected the YF antigens in both cases (see FIG. 10).

### EXAMPLE 8

Radiolabeling, Radioimmunoprecipitations and Polyacrilamide Gel Electrophoresis.

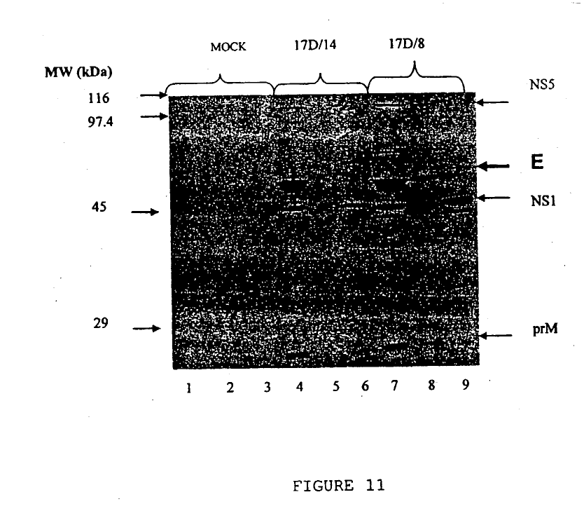

Additional evidence for correct plasmodial epitope expression on the surface of the 17D/8 virus was obtained by immunoprecipitation of metabolic labeled viral proteins. VERO cells were infected at a multiplicity of 1 PFU/cell. After 24 h incubation, the cells were labeled with [³⁵S]methionine for 1 h and lysed under nondenaturing conditions as described previously (Post et al, 1990). Cell extracts were immunoprecipitated by mouse policlonal hyperimmune ascitic fluid to YF (ATCC), two monoclonal antibodies against viral protein NS1 (Schlesinger et al, 1983) and P.falciparum repeat-specific monoclonal antibody (2A10). Immunoprecipitates were fractionated with protein A-agarose and analysed by SDS-PAGE (Laemmli, 1970). For fluorographic detection, gels were treated with sodium salicylate and autoradiographed (Chamberlain, 1979). The results are shown in FIG. 11. Immunoprecipitation profiles are obtained from protein extracts of mock-infected Vero cells (lanes 1,2,3), 17D/14 (lanes 4,5,6) or 17D/8 (lanes 7,8,9) virus-infected monolayers. These different extracts were immunoprecipitated with a murine hyperimmune serum against yellow fever virus from ATCC (lanes 1,4,7), (NANP)₃ repeat-specific monoclonal antibody or 2A10 (lanes 2,5,8) and with two monoclonal antibodies directed against NS1 (lanes 3,6,9). Molecular weight markers are shown on the left side of the figure. The gel position of some of yellow virus proteins are indicated on the right side

Both parental and recombinant viruses produced the same protein profiles when the proteins were precipitated by the mouse hyperimmune serum and 2 monoclonal antibodies to YF 17D NS1 protein (FIG. 11 lanes lanes 4, 6, 7 and 9). However, Mab 2.10 precipitated exclusively the E protein of the recombinant 1 7D/8 virus (lanes 5 and 8) consistent with the interpretation of correct expression and exposure on the surface of the epitope, as predicted by the structural analysis (FIGS. 11-13).

### EXAMPLE 9

Viral Neutralization Assay by Specific Sera

A third set of experiments to show the correct E protein surface expression of the (NANP)₃ epitope was to examine viral neutralization by specific sera. We used a plaque reduction neutralization assay in Vero cells seeded at the density of 65,000/cm² on 96-well microplates as described elsewhere (Stefano et al 1999). Neutralizing antisera included an in-house standard rhesus monkey YF-immune serum (L13) and mab 2A10. Plaque neutralization titers were calculated as the highest dilution of antibody reducing 50% of plaques of input virus, estimated by plating a mixture of virus serially diluted in fetal calf serum.

As observed in table 2 the anti YF L13 serum neutralized both viruses with the same efficiency (approximately 1:3,500). Monoclonal antibody 2A10, however only neutralized the recombinant 17D/8 virus with an extremely high titer (1:181,000), indicating the specificity of the neutralization.

TABLE 2
YF 17D virus neutralization by immune sera

| serum | YF 17 D | YF 17D/8 |
|---|---|---|
| L 13 | 1:3,715 | 1:3,388 |
| 2 A 10 | <1:79 | 1:181,970 |

In the case of the expression of the plasmodial (NANP)₃ epitope, the monoclonal antibody recognizes the linear sequence in itself as shown by the specificity of the neutralization. That suggests that the epitope is well exposed in the fg loop and its recognition is not hindered by its involvement in other viral epitope structures. It is also the first demonstration that a E protein linear epitope can be neutralizing for a flavivirus.

Fusion requires conformational changes that affect several neutralization epitopes, primarily within central domain I and domain II. These changes are apparently associated with a reorganization of the subunit interactions on the virion surface, with trimer contacts being favored in the low pH form, in contrast to dimer contacts in the native form. Interference with these structural rearrangements by antibody binding represents one mechanism that may lead to virus neutralization (Monath and Heinz, 1996). Insertion of the plasmodial epitope in a loop of domain II also led to specific viral neutralization providing further evidence for the importance of this area in viral infectivity.

### EXAMPLE 10

YF 17D/8 Recombinant Virus Attenuation

In order to demonstrate that the recombinant 17D/8 virus does not exceed its parent YF 17D virus in neuroinvasiveness and neurovirulence were carried out mouse tests.

In our analysis, groups of 16 3week-old Swiss mice were inoculated by the ic route with 3.0 log₁₀ PFU of the 17DD vaccine virus, a cDNA-derived 17D-LS3 virus and 17D/8 virus. Table 3 represents the average of two separate experiments. It is evident that the 17D/8 virus consistently kills less animals than the two other 17D viruses, 26 out of 32 for 17D/8 as opposed to 31 of 32 for the 17DD vaccine and LS3 viruses. The average survival time for animals inoculated with 17D/8 virus was also considerably longer as compared to the values obtained for the other two 17D viruses (12.5 vs 10.1 or 10.6, respectively).

TABLE 3
Mouse neurovirulence of YF17D viruses

| | IC | | |
|---|---|---|---|
| | dose | % mortality | Average survival |
| virus | (PFU) | (dead/tested) | time (days) |
| control | — | 0 (0/32) | — |
| 17DD | 10³ | 96.9 (31/32) | 10.6 |
| 17D-LS3 | 10³ | 96.9 (31/32) | 10.1 |
| 17D/8 | 10³ | 81.3 (26/32) | 12.5 |

Swiss 3 Week-Old Mice

YF 17D viruses were also examined for their capability of invading the central nervous system after peripheral (intra peritoneal, ip) inoculation into 2-5-7-9-day old Swiss mice. As shown in Table 4 below, the 17D/8 virus again behaved favourably as compared to the other 17D viruses used in being less neuroinvasive for 2 and 5-day old mice.

Epitope insertion at this site may affect the threshold of fusion-activating conformational change of this protein and it is conceivable that a slower rate of fusion may delay the extent of virus production and thereby lead to a milder infection of the host resulting in the somewhat more attenuated phenotype of the recombinant virus in the mouse model and lower extent of replication in cultured cells.

TABLE 4
Mouse Neuroinvasiveness of YF 17D Viruses

| | | 2-day | | 5-day | | 7-day | | 9-day | |
|---|---|---|---|---|---|---|---|---|---|
| | | % mortality(n | Average | % mortality(n | Average | % mortality(n dead/ | Average | % mortality(n | Average |
| virus | IP dose | dead/n tested) | survival time | dead/n tested) | survival time | n tested) | survival time | dead/n tested) | survival time |
| control | — | 0(0/12) | — | 0(0/16) | — | 0(0/7) | — | 0(0/16) | — |
| 17DD | 10³ | 75(9/12) | 11.1 | 87.5(7/8) | 11.7 | 0(0/16) | — | 0(0/14) | — |
| 17D-LS3 | 10³ | 78.5(11/14) | 11.5 | 40(4/10) | 15.5 | 21.4(3/14) | 17.3 | 5.5(1/18) | 18 |
| 17D/8 | 10³ | 12.5(1/8) | 10.0 | 0(0/19) | — | 0(0/17) | — | 0(0/16) | — |

### EXAMPLE 11

Viral Yields of YF 17D/8 in Cultured Cells.

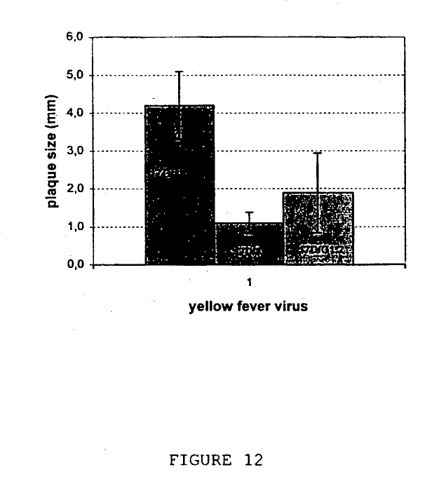

In Vero cells, the YF 17D/8 virus produced tiny plaques (1.1±0.3 mm) when compared to virus YF5.2/DD (or YF 17D/14 virus 4.20±0.9 mm) and the small plaque 17D/G1/5.2-derived virus (1.89±1.05 mm). FIG. 12 shows this data.

Viral growth curves were determined by infecting monolayers of VERO cells or primary cultures of chicken embryo fibroblasts (CEF) at m.o.i of 0.1 and 0.02 or at m.o.i of 0.1, 0.02 and 0.002, respectively. Cells were plated at density of 62,500 cell/cm² and infected 24 h later. Samples of media were collected at 24 h intervals postinfection. Viral yields were estimated by plaque titration on VERO cells.

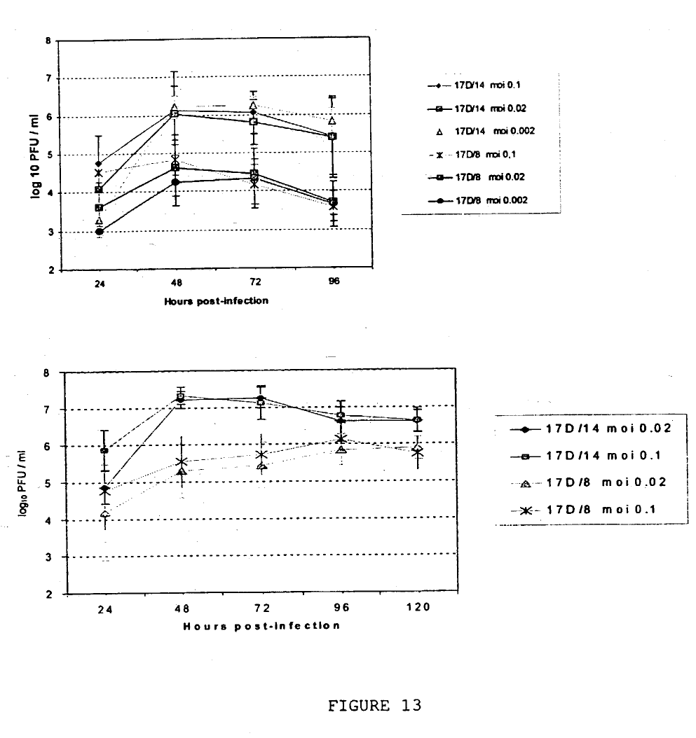

Based on single step growth curves in Vero and CEF cells differences were also observed between 17D/8 and the control virus 17D/14. In CEF cultures, which are certified for human vaccine production, both viruses produced a peak titer at 48 hrs postinfection (FIG. 13A). However maximal yields of 17D/8 reached 5×10⁴ whereas the parental virus 17D/14 without the insert reached 10⁶ PFU/ml. This difference of about 20 fold was kept until 96 hrs p.i. In Vero cells all viruses replicated better than in CEF cells but again the 17D/14-derived virus grew to 25 fold higher titers than 17D/8 (FIG. 13B).

### EXAMPLE 12

YF 17D Vaccine Virus Production in Certified Cells

According to present manufacturing efficiency and the final viral titer per human dose, it needs an initial virus titer of at least 6.0 log₁₀ PFU/ml. It is obtained a titer of 6.1 log₁₀ PFU/ml for 17D/8 in Vero cells. For vaccine manufacture, addition of stabilizer to the virus bulk reduces titer by 0.3 log₁₀ PFU/ml, filling and freeze-drying process reduces another 0.6 log₁₀ PFU/ml, a 0.6 log₁₀ PFU/ml loss in thermostability is a rule, yielding a virus preparation ready for use as a vaccine with a final titer of 3.9 log₁₀ PFU/ml. This is the minimum dosis recommended by the World Health Organization for YF vaccine in humans (WHO.1995. Requirements for Yellow Fever Vaccine).

### EXAMPLE 13

YF 17D/8 Recombinant Virus Genetic Stability

Therefore, the following experiments were carried out in order to characterize the genetic stability of the new virus (YF 17D/8) using cell cultures to propagate the virus:

Wit Regard to Insert Stability:

The integrity of the insert must be assessed by sequencing of the RT/PCR products made on RNA of culture supernatant virus. The PCR product is sequenced directly to ensure all the amino acids are in place.

It is shown for YF 17DD/204 virus that to generate vaccine-production-sized secondary seed lots at least 3 passages are necessary starting from the cloned cDNA plasmid (U.S. Pat. No. 6,171,854). Although the oligonucleotides encoding the epitopes were designed with codons more often utilized in the viral genome to avoid potential translation problems as well as instability of the inserted sequence, it is important to examine the maintenance of the insertion in the YF 17D virus genome.

For this purpose 5 series of separate passages of YF 17D/8 virus in Vero and CEF were performed with known MOI (0.1-1.0 PFU/cell). Then at selected (5th and 10th) passages the viral RNA was collected and subjected to nucleotide sequence determination (as RT-PCR products) at and around the insertion site in the E protein.

The results of these analyses in CEF and Vero cells are shown in Table 5. The insert was present in both 5th and 10th passages, suggesting its stability when the virus was passaged serially in Vero cells but not in CEF cells

TABLE 5
Genetic stability of YF 17D8 virus insert
upon serial passage in Vero and CEF cells.

| | | | |
|---|---|---|---|
| Fifth passage | insert | Tenth passage | insert |

CEF

| | | | |
|---|---|---|---|
| 17D/8 (5 a)* | − | 17D/8 (10a) | − |
| 17D/8 (5 b) | − | 17D/8 (10b) | − |

Vero

| | | | |
|---|---|---|---|
| 17D/8 (5 c) | + | 17D/8 (10 c) | + |
| 17D/8 (5 d) | + | 17D/8 (10 d) | + |
| 17D/8 (5 e) | + | 17D/8 (10 e) | + |
| 17D/8 (5 f) | + | 17D/8 (10 f) | + |
| 17D/8 (5 g) | + | 17D/8 (10 g) | + |

*The letters a and b represent two independent passages in CEF cells, whereas c, d, e, f, and g the five independent series of passages in Vero cells.

Plaque Size Analysis:

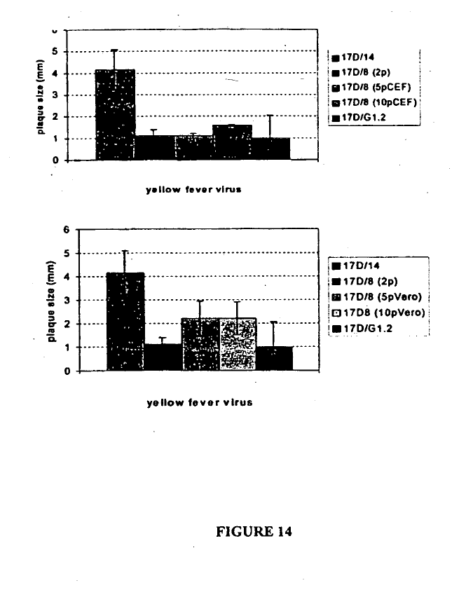

Aiming at the establishment of an in vitro marker for the recombinant virus we have studied the size of virus plaques formed on Vero cell monolayers. One of the lineages from serial passages in CEF (a) or Vero cells (b) for which sequence data on the insert region were available was also analysed for its plaque size phenotype along the passaging process. FIG. 14 displays the results of such analysis. It shows the plaque size of YF17D/8 is tiny compared to our large plaque YF 17DD/204 virus and the two small plaque 17D G1/5.2 and 17D/E200 viruses. All controls are viruses derived from cloned cDNA and have defined nucleotide sequence differences that are related to plaque size in Vero cells. In addition the plaque size displayed by both viruses is very homogeneous as expected from virus derived from cloned cDNA.

One of each lineages of the serial passages of YF 17D/8 virus in CEF (a) and Vero (b) cells were also analysed for their plaque size phenotypes along the passaging process (5th and 10^{th} passages, samples 5p and 10p, respectively). For the comparative plaque size analysis among viruses, the large plaque control, 17D/14 virus, the small plaque control, 17D/G1.2 virus, and a 17D/8 virus, representing second passage in Vero cells of the original virus recovered from RNA transfection, were used. Mean plaque diameters (mm) with the corresponding standards desviations were obtained at least from 10 different plaques of each virus.

In both cells the plaque size of serially passaged viruses tended to increase. As in Vero cell passaging there was no insert loss based on the sequencing data and plaque size phenotype in YF 17D viruses is dependent on one or more amino acid changes (A V Jabor and R Galler, unpublished) it is possible that other sequence changes took place outside the insertion area sequenced. We are presently analysing this possibility by sequencing the complete genome of YF 17D/8 virus but its expected sequence is shown comparatively to other viruses in Table 6.

TABLE 6
Comparison of YF infectious plasmid clone sequences.

| NT/gene | YFiv5.2^{a} | 17D/DD^{b} | YFiv5.2/DD^{c} | 17D/8 | 17D/1 | 17D/13 | NT [symbol] AA |
|---|---|---|---|---|---|---|---|
| 1140/E | T | C | C | T | T | T | T [symbol] Val→C [symbol] Ala |
| 1436,1437/E | G,A | A,G | A,G | G,A | G,A | G,A | G,A [symbol] D→A,G [symbol] S |
| 1946/E | T | C | C | T | C | C | T [symbol] S→C [symbol] P |
| 2219,2220/E | A,C | G,T | G,T | AC | G,T | G,T | A,C [symbol] T→G,T [symbol] V |
| 2356/E | T | T | C | T | T | T | — |
| 2602/NS1 | T | T | C | T | T | T | — |
| 2677/NS1 | C | C | T | C | C | C | — |
| 2681/NS1 | G | G | A | G | G | G | G [symbol] A→A [symbol] T |
| 8656/NS5 | A | A | C | C | A | A | — |
| 8808/NS5 | A | G | G | G | G | G | A [symbol] N→G [symbol] S |
| 9605/NS5 | G | A | A | A | A | A | A [symbol] D→G [symbol] N |
| 10454 | G | A | A | A | A | A | — |
| 10722 | G | G | A | A | G | G | — |

^{a}Rice at al (1989);
^{b}Duarte dos Santos et al, 1995;
^{c}Galler and Freire, Patent U.S. Pat. No. 6,171,854

### EXAMPLE 14

Immunogenicity in Mice

The results described above suggested that the epitope is being presented in the correct conformation and is accessible to antibodies either in solubilized native E protein as well as on the virus surface. In this example it was looked further into the immunogenicity of the recombinant 17D/8 virus by examining its capability of eliciting antibodies. It was done by immunizing BALB/c mice separately with up to 3 doses (10⁵ PFU/dose) by the intra peritoneal (IP) route given two weeks apart. Two weeks after the last dose blood was collected by intraorbital bleeding. Sera of mice were pooled and used to neutralize the homologous virus in 50% end-point plaque reduction neutralization tests. Table 7 shows these results obtained from two independent experiments. Intraperitoneal inoculation as the 3rd dose elicited an average antibody titer to the YF virus of 1:1,079 as measured by PRNT₅₀.

TABLE 7
Immunogenicity of YF 17D/8 virus in mice.

| | |
|---|---|
| Immunizing | Intra Peritoneal |

| virus | PRNT | Average PRNT |
|---|---|---|
| control | 1:13-<1:10* | <1:13 |
| 17D/E200 | 1:166-1:195 | 1:181 |
| 17D/8 | 1:741-1:417 | 1:1079 |

Reciprocal of the highest dilution of serum that resulted in 50% reduction of plaque numbers.
*These two different dilutions correspond to two set of independent experiments

### EXAMPLE 15

Construction of others Recombinant YF17D Viruses Expressing a Cytotoxic T Cell Epitope

The recombinant viruses are constructed as described in Example 6 in order to express a cytotoxic T cell epitope. The recovery of the viruses from cDNA by transfection of Vero cells was carried out as in Example 6. The resulting viruses, YF 17D/1 and 17D/13 were further passaged twice in Vero cells for the generation of working stocks. The synthetic oligonucleotide insertion at the EcoRV site of YFE200 plasmid which corresponds to the amino acid sequence depicted in Table 8 below gives rise to plasmids pYFE200/1 and pYFE200/13.. These plasmids were deposited on Dec. 21, 2000 under accesion number PTA-2858 and PTA-2854, respectively, with the American Type Culture Collection (ATCC), 10801 University Blvd., Manassas, Va.

Table 8 shows the predicted charge and isoelectric points for the epitopes alone, integrated into the fg loop and in the whole E protein context. As can be seen there is considerable variation of the net charge and the pI in each context, epitope alone, in the loop or in whole E contexts. Since the insertion region is involved in the pH-dependent conformatinal transition for fusion of the envelope to endosome membrane it is possible that this virus property could be influenced to different extents by the sequence in the epitope.

Preliminary data suggest that 17D/13 virus bearing the P.yoelii CTL epitope does not display the growth restrictions observed for the other viruses (17D/8 and 17D/1) in both CEF and Vero cells (FIG. 13). Table 8 shows the amino acid sequence and specificity of selected epitopes for insertion into YF E protein.

TABLE 8
Amino acid sequence and specificity of selected epitopes for insertion into YF E protein.

| | | | EMD | |
|---|---|---|---|---|
| | | EMD | GGNANPNANPNANPGG | |
| | YF fg loop | DYENDIEKKI IES | IES | EMD SYVPSAEQI IES |
| Antigen epitope | | CSP-CTL | CSP-B* | CSP-CTL** |
| Source | | P.falciparum | P.falciparum | P.yoelii |
| Clone | | 17D/1 | 17D/8 | 17D/13 |
| Charge/Peptide pI^{a} | — | −2.00/4.06 | 0.00/5.97 | −1.01/3.30 |
| fg loop + epitope charge/pI^{a} | −2.00/3.69 | −4.99/3.83 | −4.00/3.49 | −3.00/3.58 |
| YF E protein + epitope charge/pI^{a} | −7.4/5.83 | −9.40/5.61 | −7.40/5.83 | −8.40/5.71 |

*B, B cell
**CTL, cytotoxic T cell epitope

### EXAMPLE 16

Viral Yields of YF 17D/1 and 17D/13 in Cultured Cells.

The growth characteristics in Vero cells of YF 17D/1 and 17D/13 comparatively to other 17D virus controls including te 17D/8 recombinant expressing the humoral epitope are analysed. This example is carried out following the protocol described in Example 11.

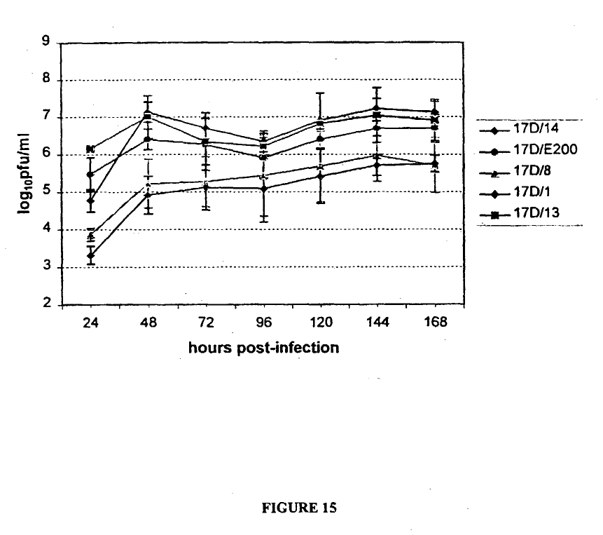

FIG. 15 shows the comparative growth curves of the 17D/14, 17D/E200 and the malaria recombinant virus 17D/8, 17D/1 and 17D/13 in Vero cells at a moi of 0.1 pfu/ml. It is evident that 17D/1 and 17D/8 viruses grow to lower titers and more slowly than our 17D/14 virus control. On the other hand the 17D/E200 and 17D/13 viruses grew as efficiently as our control virus suggesting that the insertion of SYVPSAEQI epitope was not as deleterious in this aspect as the 2 others were. The same type of growth profile in Vero cells was observed with a different MOI (0.02) and in CEF cells with both MOIs (data not shown).

There are no significant differences in the overall charge and isoelectric point of the whole E protein containing these epitopes but differences can be observed at the level of the fg loop (see Table 8) containing or not the above epitopes, the hypothesis that the charge of the epitopes may influence pH-dependent fusion and thereby lead to modified growth properties cannot be ruled out at this stage.

### EXAMPLE 17

YF 17D/1 and YF 17D/13 Recombinant Virus Genetic Stability

With Regard to Insert Stability:

As described in example 13 above for YF 17D/8 it was examined the stability of the insertion in the YF17D virus genome by nucleotide sequence analysis at the insertion site on the RNA of viruses subjected to serial passages. For this purpose each YF17D/1 and 17D/13 viruses in Vero were performed with known MOI (0.1-1.0 PFU/cell). Then at selected (5th and 10th) passages the viral RNA was collected and subjected to nucleotide sequence determination (as RT-PCR products) at and around the insertion site in the E protein.

The results of these analyses in CEF and Vero cells are shown in Table 9. The insert was present in both 5th and 10th passages, suggesting its stability when the virus was passaged serially in Vero cells but not in CEF cells. Similarly to YF17D/8 the inserted epitopes are stable throughout serial passaging of both viruses in Vero cells. These results, altogether, suggest the tolearbility of this site to the insertion of a wide variety of epitopes and endowing this system with an enormous potential for the creation of recombinant viruses expressing antigens of other pathogens in general.

TABLE 9
Genetic stability of YF 17D recombinant viruses
inserts upon serial passage in Vero cells

| Fifth passage | insert | Tenth passage | insert |
|---|---|---|---|
| 17D/1 (5 h) | + | 17D/1 (10 h) | ND |
| 17D/13 (5 i) | + | 17D/13 (10 i) | + |

ND, not determined

Plaque Size Analysis:

As shown in example 13 above the 17D/8 recombinant virus displayed a tiny plaque size phenotype as compared to our large plaque YF 17DD/204 (17D/14) virus and the two small plaque infectious cDNA-derived 17D G1/5.2 and 17D/E200 viruses. The plaque size phenotype for the new 17D/1 and 17D/13 recombinant viruses was compared to the viruses previously characterized (FIG. 15). All viruses represent second passage in Vero cells of the original virus recovered from RNA transfection.

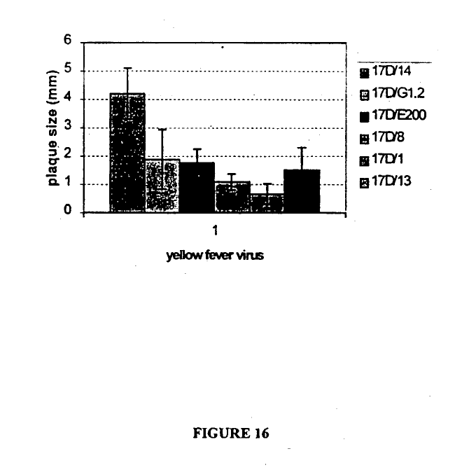

As expected from its growth profile in Vero cells (FIG. 15) the 17D/13 virus displayed a plaque size similar to 17D/E200 and 17D/G1-5.2, still small if compared to 17DD/204 (17D/14) but larger than the tiny plaques induced by 17D/1 and 17D/8 viruses (FIG. 16).

### EXAMPLE 18

YF 17D/1 and YF 17D/13 Recombinant Virus Attenuation

Although mouse neurovirulence does not predict virulence or attenuation of YF viruses for humans, it was important to demonstrate that recombinant 17D/1 and 17D/13 viruses do not exceed its parent YF 17D virus in mouse neurovirulence. The YF 17D vaccine virus displays a degree of neurotropism for mice by killing all ages of mice after intracerebral inoculation and causes usually subclinical encephalitis in monkeys (Monath, 1999).

In the present analysis, groups of 16 3 week-old Swiss mice were inoculated by the ic route with 3.0 log₁₀ PFU of the 17DD vaccine virus, 17D1, 17D/8, 17D/13 and 17D/E200 viruses. The results shown in Table 10 are representative of two separate experiments.

TABLE 10
Mouse neurovirulence of YF17D viruses

| | | % mortality | |
|---|---|---|---|
| virus | dose (PFU) | (dead/tested) | Average survival time (days) |
| control | — | 0 (1/32) | — |
| 17DD | 10³ | 96.9 (31/32) | 9.6 |
| 17D/1 | 10³ | 81.3 (26/32) | 15.6 |
| 17D/8 | 10³ | 71.9 (23/32) | 11.7 |
| 17D/13 | 10³ | 53.1 (17/32) | 15.3 |
| 17D/E200 | 10³ | 93.8 (30/32) | 11.0 |

Swiss 3 Week-Old Mice

It is evident that the 17D/8 and 17D/13 viruses consistently kill less animals than the other 17D viruses, 96.9% for 17DD and 81.3% for 17D/1 and 93.8% for 17D/E200. As observed previously the average survival time for animals inoculated with 17D/8 virus was also considerably longer as compared to the values obtained for 17DD and 17D/E200 viruses (11.7 vs 9.6 or 11.0, respectively). The 17D/1 and 17D/13 viruses killed mice at a much slower pace with ASTs of 15.4 and 15.1, respectively, but 17D/1 killed virtually all mice whereas 17D/13 was more attneuated and only killed 75%, similarly to 17D/8.

Epitope insertion at this site may affect the threshold of fusion-activated conformational change of the E protein and it is conceivable that a slower rate of fusion may delay the extent of virus production and thereby lead to a milder infection of the host resulting in the somewhat more attenuated phenotype of the recombinant virus in the mouse model and lower extent of replication in cultured cells.

The results shown here indicate that: inserts of different sizes and charges are tolerated and they are likely to influence the viral properties above. In no case, however, the recombinant virus became more neurovirulent for mice than its vaccine counterpart, further confirming the potential of using this site for the insertion of foreign epitopes and the development of new live attenuated 17D vaccines.

### EXAMPLE 19

YF 17D/13 Recombinant Virus Attenuation in Monkeys

Mouse neurovirulence had not been considered as a reliable marker for the attenuation of YF viruses for humans until recently when Monath et al (2002) studying the attenuation of chimeric YF 17D-Japanese encephalitis viruses have proposed it. However, the final definition for the attenuation of YF viruses must corn from internationally accepted monkey neurovirulence tests recommended by the World Health Organization for the characterization of new YF seed and vaccine viruses (WHO, 1998). Here, quantitative pathological examination of brain and spinal cord tissue provides a sensitive method for distinguishing strains of the same virus with subtle differences in neurovirulence (Levenbook et al, 1987; Marchevsky et al, 1995; WHO 1998).

We have examined the attenuation of YF 17D/13 virus, the most attenuated in our mouse model, in the current monkey neurovirulence test (WHO, 1998) to establish its preclinical safety. Here twenty rhesus monkeys, obtained from the colony at the Oswaldo Cruz Foundation, weighing from 2.7 to 6.4 kg, being 14 females and 6 males were equally distributed between two groups of 10. One group of 10 monkeys received a single inoculation of YF 17DD secondary seed lot virus (BioManguinhos-Fiocruz) into the frontal lobe of the brain and a second group of ten animals received the experimental YF 17D/13 virus. Each 0.25 ml of inoculum contained 6.53 Log₁₀ PFU/ml PFU of 17DD virus or 6.62 Log₁₀ PFU/ml of 17D/13. Monkeys were observed for 30 days. Records of clinical observation were obtained using the following signs: grade 1=rough coat, not eating; grade 2=high-pitched voice, inactive, slow moving; grade 3=shaky movements, tremors, uncoordinated movement, limb weakness; grade 4=inability to stand, limb paralysis or death. A monkey that dies receive the score “4”from the day of death until day 30.

Viremia levels were measured on days 2, 4 and 6 after inoculation by plaquing in Vero cells samples of monkey sera. Seroconversion was measured by the appearance of neutralizing antibodies on day 31. On this day, animals were euthanized and a full necropsy was performed. Brains and spinal cord were examined and scored as indicated (WHO, 1998). Five levels of the brain and six levels of each of the lumbar and cervical enlargements were examined. Brain levels included: Block I, the corpus striatum at the level of the optic chiasma; block II, the thalamus at the level of the mamillary bodies; block III, the mesencephalon at the level of the superior colliculi; block IV, the pons and cerebellum at the level of the superior olives; block V, the medulla oblongata at the mid-level of the inferior olives (WHO, 1998). Numerical scores were given to each hemisection of the cord and to structures in each hemisection of the brain. Lesions were scored according to the following grading system: 1, (minimal), 1-3 small, focal inflammatory infiltrates, a few neurons may be changed or lost; 2 (moderate), more extensive focal inflammatory infiltrates, neuronal changes or loss affects no more than one third of neurons; 3, (severe), neuronal changes or loss of 33-90% of neurons, with moderate focal or diffuse inflammatory infiltration; 4, (overwhelming), more than 90% of neurons are changed or lost, with variable, but frequently severe, inflammatory infiltration.

All neuropathological evaluations, clinical and histological, were done by a single, experienced investigator who was blinded to the treatment code.

Three separate scores were calculated for each monkey: discriminator areas only, target areas only, and discriminator plus target areas (Levenbook et al, 1987). The target area is the substantia nigra where all 17D viruses replicate whereas the discriminator areas include the caudate nucleus, globus pallidus, putamen, anterior and medial thalamic nucleus, lateral thalamic nucleus, cervical and lumbar enlargements and only neurovirulent viruses induce significant neuronal loss. A final neurovirulence score is given by the combination of the scores of both areas (combined score).

Viremia

Table 11 displays the data on viremia recorded for monkeys inoculated with each virus. In the experimental infection of rhesus monkeys by the intracerebral route with 17DD and 17D/13 viruses, monkey serum viremia differs between the viruses as only 5 animals were viremic at any given day (2-4-6) after inoculation with the latter whereas the former induced viremia in 8 out of 10 animals. Viremia was most prevalent in both groups at the 4^{th} day post infection when 5 out of 10 monkeys showed measurable circulating virus. Monkeys that received 17D/13 virus also presented less viremia days (5) as compared to 17DD (9). In addition the highest peak of viremia for 17D/13 virus was 1.44 log₁₀ PFU/ml whereas for 17DD was about 10 fold higher (2.42 log₁₀ PFU/ml). However, both viruses are well below the limits established by WHO (1998).

The definition put forward by WHO (1998) of viscerotropism of 17D virus limits the amount of circulating virus to below 500 mouse LD₅₀/0.03 mL for all (10 out of 10) sera and ≧100 LD₅/0.03 mL in one out of 10 monkey sera at 1:10 dilution. In this regard, the highest viremia observed was 2.42 log₁₀ PFU/ml for monkey 810, inoculated with 17DD virus. That corresponds to 1.64 LD₅₀/0.03 mL, therefore, well below the established linits. In addition, the range of titers observed here is similar to that observed for rhesus monkeys inoculated with attenuated 17D/JE SA-14-14-2; 17D-den2 chimeric and 17D-204 viruses (Monath et al, 2000; 2002; Guirakhoo et al, 2000).

Clinical Score

Table 11 displays the individual clinical scores after the 30-day observation period. This score is the average of the values given at each day during this period. It is shown in Table 11 that only 2 monkeys (6U and 46) inoculated with 17D/13 virus displayed any clinical signs as compared to 5 monkeys inoculated with 17DD virus (114, 240, 303, 810 and O31). The fact that several animals displayed viremia and all specifically seroconverted to YF in plaque reduction neutralization tests (Table 11) confirm that animals were indeed infected by the respective virus inoculated. From the monkeys inoculated with 17DD virus, monkeys 114, 810 and 240 had the highest viremias but yet minimal scores (0.07, 0.14 and 0.64, respectively). For 17D/13, monkey 253 showed no clinical signs and yet had the highest viremia in the group (Table 11).

The overall clinical score for each group is given in Table 12 along with the histological scores. The mean clinical score for YF 17DD vaccine virus was 0.11 whereas for 17D/13 it was 0.17. A somewhat higher score for the latter is due to a single outlyer monkey that presented some signs of encephalitis (monkey 6U). That particular monkey showed no viremia, did show seroconversion to YF and did not present histological alterations in the central nervous system compatible with the clinical picture (Table 12). Lastly, the scores for both viruses are comparable to clincal scores observed by other groups for other 17D viruses (Marchevsky et al, 1995; Monath et al, 2000; 2002) suggesting the attenuation of YF 17D/13 virus.

Histological Score

All twenty rhesus monkeys inoculated i.c with 17DD and 17D/13 viruses developed histological lesions (Table 12) in the central nervous system (CNS). For all animals, there were no abnormalities in any extraneural organs that could suggest damage or impaired function. None of the animals developed any histological lesions the liver, kidney, adrenals, heart, spleen, lungs. As proposed by Levenbook et al (1987) the target area in rhesus monkeys CNS for several vaccine viruses is the substantia nigra. In this study the substantia nigra presented with the highest histological scores for the monkeys inoculated i.c. with both viruses. Based on the individual values shown in Table 12, 17DD virus had an average score in this area of 1.75, and it was 1.40 for 17D/13 virus. In five complete neurovirulence tests for 17DD 102/84 seed lot virus the average target area score was 1.49 (R S Marchevsky and R Galler, in preparation).

Among the discriminator areas in the CNS, the putamen, globus pallidus and nucleus caudatus were the areas more affected but the lesion scores were never above 2 with any of the viruses. Monkey 6U inoculated with 17D/13 virus and which presented the highest clinical score (1.00) among the 20 animals showed the third lowest score in the discriminatory areas (0.33; Table 12). The average discriminator area score for 17DD virus was 0.78 and 0.53 for 17D/13 virus, values which are close to each other and to the average value observed for 102/84 across five other full neurovirulence tests (0.67; R S Marchevsky and R Galler, in preparation).

The degree of neurovirulence of a given virus is the average of combined target/discriminator areas scores of all the monkeys. For 17DD virus this combined score was 1.21 whereas for 17D/13 it was 0.96. The values for the combined neurovirulence scores in five complete tests with 102/84 virus varied between 0.96 and 1.37 with an average of 1.07. For YF 17D-204 virus the target, discriminatory and combined areas scores were 1.63, 0.71 and 1.17, respectively (Monath et al, 2002).

The observation of minimal clinical signs of infection, no extraneural organ alterations, CNS histological scores of animals inoculated i.c. within the expected range and limited viremia suggest an attenuated phenotype for the YF 17D/13 recombinant virus. The fact that all animals mounted a strong neutralizing anti-YF antibody response to 17D/13 virus suggests that also in nonhuman primates the recombinant virus has maintained its immunogenicity despite the epitope insertion in its envelope E protein.

The results shown here indicate that: inserts of different sizes and charges are tolerated and they are likely to influence viral properties such as low pH-dependent viral envelope fusion. In no case, however, the recombinant viruses tested became more neurovirulent for mice than its vaccine counterpart. The testing of one such recombinant virus (17D/13) for monkey neurovirulence also suggested that insertion at the 17D E protein fg loop does not compromise its atttenuated phenotype further confirming the potential use of this site for the insertion of foreign epitopes and the development of new live attenuated 17D vaccines.

TABELA 11
Recorded parameters for the monkey
neurovirulence test of YF 17D viruses

| | | | | | | Combined | | |
|---|---|---|---|---|---|---|---|---|
| | | Viremia | | | Clinical | histological | Seroconversion | |
| Virus | Monkey | 2nd | 4th | 6th | score | score | Pre | Post |
| 17DD | 114 | <0.6 | 1.83 | <0.6 | 0.07 | 1.10 | <447 | 95471 |
| | 116 | 0.6 | <0.6 | <0.6 | 0 | 0.96 | <447 | 30124 |
| | 159 | <0.6 | <0.6 | 1.08 | 0 | 0.35 | <447 | 41210 |
| | 162 | 1.20 | 1.20 | <0.6 | 0 | 1.51 | <447 | 35872 |
| | 240 | 1.68 | <0.6 | <0.6 | 0.64 | 1.39 | <447 | 141947 |
| | 303 | <0.6 | <0.6 | <0.6 | 0.17 | 1.44 | <174 | 46773 |
| | 810 | 0.9 | 2.42 | <0.6 | 0.14 | 1.38 | <174 | 72028 |
| | 934 | <0.6 | <0.6 | <0.6 | 0 | 1.32 | <100 | >100000 |
| | 4U | <0.6 | 1.20 | <0.6 | 0 | 1.45 | <100 | >100000 |
| | O31 | <0.6 | 0.9 | <0.6 | 0.10 | 1.26 | <100 | 16999 |
| 17D/13 | 178 | <0.6 | 0.6 | <0.6 | 0 | 0.79 | <447 | 50646 |
| | 253 | <0.6 | 1.44 | <0.6 | 0 | 0.61 | <174 | 3770 |
| | 423 | <0.6 | 0.6 | <0.6 | 0 | 0.69 | <174 | 32393 |
| | 520 | <0.6 | 0.9 | <0.6 | 0 | 1.09 | <174 | 13375 |
| | 540 | <0.6 | <0.6 | <0.6 | 0 | 0.73 | <174 | 8511 |
| | 558 | <0.6 | 0.9 | <0.6 | 0 | 1.28 | <174 | 646 |
| | 4^{A} | <0.6 | <0.6 | <0.6 | 0 | 1.33 | <100 | 40179 |
| | 6U | <0.6 | <0.6 | <0.6 | 1.00 | 0.67 | <100 | 10448 |
| | 46 | <0.6 | <0.6 | <0.6 | 0.67 | 1.42 | <100 | 7154 |
| | T73 | <0.6 | <0.6 | <0.6 | 0 | 1.02 | <100 | 1136 |

TABLE 12
Neurovirulence of YF 17D viruses for rhesus monkeys

| | | Clinical | Disriminator | | Combined |
|---|---|---|---|---|---|
| Virus | Monkeys | score | areas | Target area | score |
| 17DD | 114 | 0.07 | 0.7 | 1.5 | 1.10 |
| | 116 | 0 | 0.42 | 1.5 | 0.96 |
| | 159 | 0 | 0.21 | 0.5 | 0.35 |
| | 162 | 0 | 1.03 | 2 | 1.51 |
| | 240 | 0.64 | 0.78 | 2 | 1.39 |
| | 303 | 0.17 | 0.88 | 2 | 1.44 |
| | 810 | 0.14 | 0.76 | 2 | 1.38 |
| | 934 | 0 | 0.64 | 2 | 1.32 |
| | 4U | 0 | 0.91 | 2 | 1.45 |
| | O31 | 0.1 | 0.52 | 2 | 1.26 |
| Mean | | 0.112 | 0.78 | 1.75 | 1,216 |
| 17D/13 | 178 | 0 | 0.59 | 1 | 0.79 |
| | 253 | 0 | 0.23 | 1 | 0.61 |
| | 423 | 0 | 0.38 | 1 | 0.69 |
| | 520 | 0 | 0.69 | 1.5 | 1.09 |
| | 540 | 0 | 0.47 | 1 | 0.73 |
| | 558 | 0 | 0.57 | 2 | 1.28 |
| | 4^{A} | 0 | 0.67 | 2 | 1.33 |
| | 6U | 1 | 0.33 | 1 | 0.67 |
| | 46 | 0.67 | 0.84 | 2 | 1.42 |
| | T73 | 0 | 0.54 | 1.5 | 1.02 |
| Mean | | 0.167 | 0.53 | 1.40 | 0,963 |

### LIST SEQUENCING

I.a) Applicant: Fundacão Oswaldo Cruz

I.b) Address: Av. Brasil, 4365, Castelo Mourisco, sala 05, Manguinhos—21045-900—Rio de Janeiro—RJ

II) Invention: Use of Flavivirus for the expression of protein epitopes and development of new live attenuated vaccine virus to immunize against flavivirus and other infectious agents.

III) Number of Sequences: 01 (one).

1) INFORMATION FOR SEQ ID NO:1

I) Sequence characteristics:

I.a) length: 10862 base pairs

I.b) type: nucleic acid

I.c) strandedness: single

I.d) topology: linear

I.e) molecule type: cDNA

I.f) original source: individual isolate: Yfiv5.2/DD

I.g) sequence description: SEQ ID NO:1:

| | |
|---|---|
| 1 | AGTAAATCCT GTGTGCTAAT TGAGGTGCAT TGGTCTGCAA ATCGAGTTGC |
| 51 | TAGGCAATAA ACACATTTGG ATTAATTTTA ATCGTTCGTT GAGCGATTAG |
| 101 | CAGAGAACTG ACCAGAACAT GTCTGGTCGT AAAGCTCAGG GAAAAACCCT |
| 151 | GGGCGTCAAT ATGGTACGAC GAGGAGTTCG CTCCTTGTCA AACAAAATAA |
| 201 | AACAAAAAAC AAAACAAATT GGAAACAGAC CTGGACCTTC AAGAGGTGTT |
| 251 | CAAGGATTTA TCTTTTTCTT TTTGTTCAAC ATTTTGACTG GAAAAAAGAT |
| 301 | CACAGCCCAC CTAAAGAGGT TGTGGAAAAT GCTGGACCCA AGACAAGGCT |
| 351 | TGGCTGTTCT AAGGAAAGTC AAGAGAGTGG TGGCCAGTTT GATGAGAGGA |
| 401 | TTGTCCTCAA GGAAACGCCG TTCCCATGAT GTTCTGACTG TGCAATTCCT |
| 451 | AATTTTGGGA ATGCTGTTGA TGACGGGTGG AGTGACCTTG GTGCGGAAAA |
| 501 | ACAGATGGTT GCTCCTAAAT GTGACATCTG AGGACCTCGG GAAAACATTC |
| 551 | TCTGTGGGCA CAGGCAACTG CACAACAAAC ATTTTGGAAG CCAAGTACTG |
| 601 | GTGCCCAGAC TCAATGGAAT ACAACTGTCC CAATCTCAGT CCAAGAGAGG |
| 651 | AGCCAGATGA CATTGATTGC TGGTGCTATG GGGTGGAAAA CGTTAGAGTC |
| 701 | GCATATGGTA AGTGTGACTC AGCAGGCAGG TCTAGGAGGT CAAGAAGGGC |
| 751 | CATTGACTTG CCTACGCATG AAAACCATGG TTTGAAGACC CGGCAAGAAA |
| 801 | AATGGATGAC TGGAAGAATG GGTGAAAGGC AACTCCAAAA GATTGAGAGA |
| 851 | TGGTTCGTGA GGAACCCCTT TTTTGCAGTG ACGGCTCTGA CCATTGCCTA |
| 901 | CCTTGTGGGA AGCAACATGA CGCAACGAGT CGTGATTGCC CTACTGGTCT |
| 951 | TGGCTGTTGG TCCGGCCTAC TCAGCTCACT GCATTGGAAT TACTGACAGG |
| 1001 | GATTTCATTG AGGGGGTGCA TGGAGGAACT TGGGTTTCAG CTACCCTGGA |
| 1051 | GCAAGACAAG TGTGTCACTG TTATGGCCCC TGACAAGCCT TCATTGGACA |
| 1101 | TCTCACTAGA GACAGTAGCC ATTGATAGAC CTGCTGAGGC GAGGAAAGTG |
| 1151 | TGTTACAATG CAGTTCTCAC TCATGTGAAG ATTAATGACA AGTGCCCCAG |
| 1201 | CACTGGAGAG GCCCACCTAG CTGAAGAGAA CGAAGGGGAC AATGCGTGCA |
| 1251 | AGCGCACTTA TTCTGATAGA GGCTGGGGCA ATGGCTGTGG CCTATTTGGG |
| 1301 | AAAGGGAGCA TTGTGGCATG CGCCAAATTC ACTTGTGCCA AATCCATGAG |
| 1351 | TTTGTTTGAG GTTGATCAGA CCAAAATTCA GTATGTCATC AGAGCACAAT |
| 1401 | TGCATGTAGG GGCCAAGCAG GAAAATTGGA ATACCAGCAT TAAGACTCTC |
| 1451 | AAGTTTGATG CCCTGTCAGG CTCCCAGGAA GTCGAGTTCA TTGGGTATGG |
| 1501 | AAAAGCTACA CTGGAATGCC AGGTGCAAAC TGCGGTGGAC TTTGGTAACA |
| 1551 | GTTACATCGC TGAGATGGAA ACAGAGAGCT GGATAGTGGA CAGACAGTGG |
| 1601 | GCCCAGGACT TGACCCTGCC ATGGCAGAGT GGAAGTGGCG GGGTGTGGAG |
| 1651 | AGAGATGCAT CATCTTGTCG AATTTGAACC TCCGCATGCC GCCACTATCA |
| 1701 | GAGTACTGGC CCTGGGAAAC CAGGAAGGCT CCTTGAAAAC AGCTCTTACT |
| 1751 | GGCGCAATGA GGGTTACAAA GGACACAAAT GACAACAACC TTTACAAACT |
| 1801 | ACATGGTGGA CATGTTTCTT GCAGAGTGAA ATTGTCAGCT TTGACACTCA |
| 1851 | AGGGGACATC CTACAAAATA TGCACTGACA AAATGTTTTT TGTCAAGAAC |
| 1901 | CCAACTGACA CTGGCCATGG CACTGTTGTG ATGCAGGTGA AAGTGCCAAA |
| 1951 | AGGAGCCCCC TGCAGGATTC CAGTGATAGT AGCTGATGAT CTTACAGCGG |
| 2001 | CAATCAATAA AGGCATTTTG GTTACAGTTA ACCCCATCGC CTCAACCAAT |
| 2051 | GATGATGAAG TGCTGATTGA GGTGAACCCA CCTTTTGGAG ACAGCTACAT |
| 2101 | TATCGTTGGG AGAGGAGATT CACGTCTCAC TTACCAGTGG CACAAAGAGG |
| 2151 | GAAGCTCAAT AGGAAAGTTG TTCACTCAGA CCATGAAAGG CGTGGAACGC |
| 2201 | CTGGCCGTCA TGGGAGACGT CGCCTGGGAT TTCAGCTCCG CTGGAGGGTT |
| 2251 | CTTCACTTCG GTTGGGAAAG GAATTCATAC GGTGTTTGGC TCTGCCTTTC |
| 2301 | AGGGGCTATT TGGCGGCTTG AACTGGATAA CAAAGGTCAT CATGGGGGCG |
| 2351 | GTACTCATAT GGGTTGGCAT CAACACAAGA AACATGACAA TGTCCATGAG |
| 2401 | CATGATCTTG GTAGGAGTGA TCATGATGTT TTTGTCTCTA GGAGTTGGGG |
| 2451 | CGGATCAAGG ATGCGCCATC AACTTTGGCA AGAGAGAGCT CAAGTGCGGA |
| 2501 | GATGGTATCT TCATATTTAG AGACTCTGAT GACTGGCTGA ACAAGTACTC |
| 2551 | ATACTATCCA GAAGATCCTG TGAAGCTTGC ATCAATAGTG AAAGCCTCTT |
| 2601 | TCGAAGAAGG GAAGTGTGGC CTAAATTCAG TTGACTCCCT TGAGCATGAG |
| 2651 | ATGTGGAGAA GCAGGGCAGA TGAGATTAAT ACCATTTTTG AGGAAAACGA |
| 2701 | GGTGGACATT TCTGTTGTCG TGCAGGATCC AAAGAATGTT TACCAGAGAG |
| 2751 | GAACTCATCC ATTTTCCAGA ATTCGGGATG GTCTGCAGTA TGGTTGGAAG |
| 2801 | ACTTGGGGTA AGAACCTTGT GTTCTCCCCA GGGAGGAAGA ATGGAAGCTT |
| 2851 | CATCATAGAT GGAAAGTCCA GGAAAGAATG CCCGTTTTCA AACCGGGTCT |
| 2901 | GGAATTCTTT CCAGATAGAG GAGTTTGGGA CGGGAGTGTT CACCACACGC |
| 2951 | GTGTACATGG ACGCAGTCTT TGAATACACC ATAGACTGCG ATGGATCTAT |
| 3001 | CTTGGGTGCA GCGGTGAACG GAAAAAAGAG TGCCCATGGC TCTCCAACAT |
| 3051 | TTTGGATGGG AAGTCATGAA GTAAATGGGA CATGGATGAT CCACACCTTG |
| 3101 | GAGGCATTAG ATTACAAGGA GTGTGAGTGG CCACTGACAC ATACGATTGG |
| 3151 | AACATCAGTT GAAGAGAGTG AAATGTTCAT GCCGAGATCA ATCGGAGGCC |
| 3201 | CAGTTAGCTC TCACAATCAT ATCCCTGGAT ACAAGGTTCA GACGAACGGA |
| 3251 | CCTTGGATGC AGGTACCACT AGAAGTGAAG AGAGAAGCTT GCCCAGGGAC |
| 3301 | TAGCGTGATC ATTGATGGCA ACTGTGATGG ACGGGGAAAA TCAACCAGAT |
| 3351 | CCACCACGGA TAGCGGGAAA GTTATTCCTG AATGGTGTTG CCGCTCCTGC |
| 3401 | ACAATGCCGC CTGTGAGCTT CCATGGTAGT GATGGGTGTT GGTATCCCAT |
| 3451 | GGAAATTAGG CCAAGGAAAA CGCATGAAAG CCATCTGGTG CGCTCCTGGG |
| 3501 | TTACAGCTGG AGAAATACAT GCTGTCCCTT TTGGTTTGGT GAGCATGATG |
| 3551 | ATAGCAATGG AAGTGGTCCT AAGGAAAAGA CAGGGACCAA AGCAAATGTT |
| 3601 | GGTTGGAGGA GTAGTGCTCT TGGGAGCAAT GCTGGTCGGG CAAGTAACTC |
| 3651 | TCCTTGATTT GCTGAAACTC ACAGTGGCTG TGGGATTGCA TTTCCATGAG |
| 3701 | ATGAACAATG GAGGAGACGC CATGTATATG GCGTTGATTG CTGCCTTTTC |
| 3751 | AATCAGACCA GGGCTGCTCA TCGGCTTTGG GCTCAGGACC CTATGGAGCC |
| 3801 | CTCGGGAACG CCTTGTGCTG ACCCTAGGAG CAGCCATGGT GGAGATTGCC |
| 3851 | TTGGGTGGCG TGATGGGCGG CCTGTGGAAG TATCTAAATG CAGTTTCTCT |
| 3901 | CTGCATCCTG ACAATAAATG CTGTTGCTTC TAGGAAAGCA TCAAATACCA |
| 3951 | TCTTGCCCCT CATGGCTCTG TTGACACCTG TCACTATGGC TGAGGTGAGA |
| 4001 | CTTGCCGCAA TGTTCTTTTG TGCCGTGGTT ATCATAGGGG TCCTTCACCA |
| 4051 | GAATTTCAAG GACACCTCCA TGCAGAAGAC TATACCTCTG GTGGCCCTCA |
| 4101 | CACTCACATC TTACCTGGGC TTGACACAAC CTTTTTTGGG CCTGTGTGCA |
| 4151 | TTTCTGGCAA CCCGCATATT TGGGCGAAGG AGTATCCCAG TGAATGAGGC |
| 4201 | ACTCGCAGCA GCTGGTCTAG TGGGAGTGCT GGCAGGACTG GCTTTTCAGG |
| 4251 | AGATGGAGAA CTTCCTTGGT CCGATTGCAG TTGGAGGACT CCTGATGATG |
| 4301 | CTGGTTAGCG TGGCTGGGAG GGTGGATGGG CTAGAGCTCA AGAAGCTTGG |
| 4351 | TGAAGTTTCA TGGGAAGAGG AGGCGGAGAT CAGCGGGAGT TCCGCCCGCT |
| 4401 | ATGATGTGGC ACTCAGTGAA CAAGGGGAGT TCAAGCTGCT TTCTGAAGAG |
| 4451 | AAAGTGCCAT GGGACCAGGT TGTGATGACC TCGCTGGCCT TGGTTGGGGC |
| 4501 | TGCCCTCCAT CCATTTGCTC TTCTGCTGGT CCTTGCTGGG TGGCTGTTTC |
| 4551 | ATGTCAGGGG AGCTAGGAGA AGTGGGGATG TCTTGTGGGA TATTCCCACT |
| 4601 | CCTAAGATCA TCGAGGAATG TGAACATCTG GAGGATGGGA TTTATGGCAT |
| 4651 | ATTCCAGTCA ACCTTCTTGG GGGCCTCCCA GCGAGGAGTG GGAGTGGCAC |
| 4701 | AGGGAGGGGT GTTCCACACA ATGTGGCATG TCACAAGAGG AGCTTTCCTT |
| 4751 | GTCAGGAATG GCAAGAAGTT GATTCCATCT TGGGCTTCAG TAAAGGAAGA |
| 4801 | CCTTGTCGCC TATGGTGGCT CATGGAAGTT GGAAGGCAGA TGGGATGGAG |
| 4851 | AGGAAGAGGT CCAGTTGATC GCGGCTGTTC CAGGAAAGAA CGTGGTCAAC |
| 4901 | GTCCAGACAA AACCGAGCTT GTTCAAAGTG AGGAATGGGG GAGAAATCGG |
| 4951 | GGCTGTCGCT CTTGACTATC CGAGTGGCAC TTCAGGATCT CCTATTGTTA |
| 5001 | ACAGGAACGG AGAGGTGATT GGGCTGTACG GCAATGGCAT CCTTGTCGGT |
| 5051 | GACAACTCCT TCGTGTCCGC CATATCCCAG ACTGAGGTGA AGGAAGAAGG |
| 5101 | AAAGGAGGAG CTCCAAGAGA TCCCGACAAT GCTAAAGAAA GGAATGACAA |
| 5151 | CTGTCCTTGA TTTTCATCCT GGAGCTGGGA AGACAAGACG TTTCCTCCCA |
| 5201 | CAGATCTTGG CCGAGTGCGC ACGGAGACGC TTGCGCACTC TTGTGTTGGC |
| 5251 | CCCCACCAGG GTTGTTCTTT CTGAAATGAA GGAGGCTTTT CACGGCCTGG |
| 5301 | ACGTGAAATT CCACACACAG GCTTTTTCCG CTCACGGCAG CGGGAGAGAA |
| 5351 | GTCATTGATG CCATGTGCCA TGCCACCCTA ACTTACAGGA TGTTGGAACC |
| 5401 | AACTAGGGTT GTTAACTGGG AAGTGATCAT TATGGATGAA GCCCATTTTT |
| 5451 | TGGATCCAGC TAGCATAGCC GCTAGAGGTT GGGCAGCGCA CAGAGCTAGG |
| 5501 | GCAAATGAAA GTGCAACAAT CTTGATGACA GCCACACCGC CTGGGACTAG |
| 5551 | TGATGAATTT CCACATTCAA ATGGTGAAAT AGAAGATGTT CAAACGGACA |
| 5601 | TACCCAGTGA GCCCTGGAAC ACAGGGCATG ACTGGATCCT AGCTGACAAA |
| 5651 | AGGCCCACGG CATGGTTCCT TCCATCCATC AGAGCTGCAA ATGTCATGGC |
| 5701 | TGCCTCTTTG CGTAAGGCTG GAAAGAGTGT GGTGGTCCTG AACAGGAAAA |
| 5751 | CCTTTGAGAG AGAATACCCC ACGATAAAGC AGAAGAAACC TGACTTTATA |
| 5801 | TTGGCCACTG ACATAGCTGA AATGGGAGCC AACCTTTGCG TGGAGCGAGT |
| 5851 | GCTGGATTGC AGGACGGCTT TTAAGCCTGT GCTTGTGGAT GAAGGGAGGA |
| 5901 | AGGTGGCAAT AAAAGGGCCA CTTCGTATCT CCGCATCCTC TGCTGCTCAA |
| 5951 | AGGAGGGGGC GCATTGGGAG AAATCCCAAC AGAGATGGAG ACTCATACTA |
| 6001 | CTATTCTGAG CCTACAAGTG AAAATAATGC CCACCACGTC TGCTGGTTGG |
| 6051 | AGGCCTCAAT GCTCTTGGAC AACATGGAGG TGAGGGGTGG AATGGTCGCC |
| 6101 | CCACTCTATG GCGTTGAAGG AACTAAAACA CCAGTTTCCC CTGGTGAAAT |
| 6151 | GAGACTGAGG GATGACCAGA GGAAAGTCTT CAGAGAACTA GTGAGGAATT |
| 6201 | GTGACCTGCC CGTTTGGCTT TCGTGGCAAG TGGCCAAGGC TGGTTTGAAG |
| 6251 | ACGAATGATC GTAAGTGGTG TTTTGAAGGC CCTGAGGAAC ATGAGATCTT |
| 6301 | GAATGACAGC GGTGAAACAG TGAAGTGCAG GGCTCCTGGA GGAGCAAAGA |
| 6351 | AGCCTCTGCG CCCAAGGTGG TGTGATGAAA GGGTGTCATC TGACCAGAGT |
| 6401 | GCGCTGTCTG AATTTATTAA GTTTGCTGAA GGTAGGAGGG GAGCTGCTGA |
| 6451 | AGTGCTAGTT GTGCTGAGTG AACTCCCTGA TTTCCTGGCT AAAAAAGGTG |
| 6501 | GAGAGGCAAT GGATACCATC AGTGTGTTTC TCCACTCTGA GGAAGGCTCT |
| 6551 | AGGGCTTACC GCAATGCACT ATCAATGATG CCTGAGGCAA TGACAATAGT |
| 6601 | CATGCTGTTT ATACTGGCTG GACTACTGAC ATCGGGAATG GTCATCTTTT |
| 6651 | TCATGTCTCC CAAAGGCATC AGTAGAATGT CTATGGCGAT GGGCACAATG |
| 6701 | GCCGGCTGTG GATATCTCAT GTTCCTTGGA GGCGTCAAAC CCACTCACAT |
| 6751 | CTCCTATATC ATGCTCATAT TCTTTGTCCT GATGGTGGTT GTGATCCCCG |
| 6801 | AGCCAGGGCA ACAAAGGTCC ATCCAAGACA ACCAAGTGGC ATACCTCATT |
| 6851 | ATTGGCATCC TGACGCTGGT TTCAGCGGTG GCAGCCAACG AGCTAGGCAT |
| 6901 | GCTGGAGAAA ACCAAAGAGG ACCTCTTTGG GAAGAAGAAC TTAATTCCAT |
| 6951 | CTAGTGCTTC ACCCTGGAGT TGGCCGGATC TTGACCTGAA GCCAGGAGCT |
| 7001 | GCCTGGACAG TGTACGTTGG CATTGTTACA ATGCTCTCTC CAATGTTGCA |
| 7051 | CCACTGGATC AAAGTCGAAT ATGGCAACCT GTCTCTGTCT GGAATAGCCC |
| 7101 | AGTCAGCCTC AGTCCTTTCT TTCATGGACA AGGGGATACC ATTCATGAAG |
| 7151 | ATGAATATCT CGGTCATAAT GCTGCTGGTC AGTGGCTGGA ATTCAATAAC |
| 7201 | AGTGATGCCT CTGCTCTGTG GCATAGGGTG CGCCATGCTC CACTGGTCTC |
| 7251 | TCATTTTACC TGGAATCAAA GCGCAGCAGT CAAAGCTTGC ACAGAGAAGG |
| 7301 | GTGTTCCATG GCGTTGCCAA GAACCCTGTG GTTGATGGGA ATCCAACAGT |
| 7351 | TGACATTGAG GAAGCTCCTG AAATGCCTGC CCTTTATGAG AAGAAACTGG |
| 7401 | CTCTATATCT CCTTCTTGCT CTCAGCCTAG CTTCTGTTGC CATGTGCAGA |
| 7451 | ACGCCCTTTT CATTGGCTGA AGGCATTGTC CTAGCATCAG CTGCCTTAGG |
| 7501 | GCCGCTCATA GAGGGAAACA CCAGCCTTCT TTGGAATGGA CCCATGGCTG |
| 7551 | TCTCCATGAC AGGAGTCATG AGGGGGAATC ACTATGCTTT TGTGGGAGTC |
| 7601 | ATGTACAATC TATGGAAGAT GAAAACTGGA CGCCGGGGGA GCGCGAATGG |
| 7651 | AAAAACTTTG GGTGAAGTCT GGAAGAGGGA ACTGAATCTG TTGGACAAGC |
| 7701 | GACAGTTTGA GTTGTATAAA AGGACCGACA TTGTGGAGGT GGATCGTGAT |
| 7751 | ACGGCACGCA GGCATTTGGC CGAAGGGAAG GTGGACACCG GGGTGGCGGT |
| 7801 | CTCCAGGGGG ACCGCAAAGT TAAGGTGGTT CCATGAGCGT GGCTATGTCA |
| 7851 | AGCTGGAAGG TAGGGTGATT GACCTGGGGT GTGGCCGCGG AGGCTGGTGT |
| 7901 | TACTACGCTG CTGCGCAAAA GGAAGTGAGT GGGGTCAAAG GATTTACTCT |
| 7951 | TGGAAGAGAC GGCCATGAGA AACCCATGAA TGTGCAAAGT CTGGGATGGA |
| 8001 | ACATCATCAC CTTCAAGGAC AAAACTGATA TCCACCGCCT AGAACCAGTG |
| 8051 | AAATGTGACA CCCTTTTGTG TGACATTGGA GAGTCATCAT CGTCATCGGT |
| 8101 | CACAGAGGGG GAAAGGACCG TGAGAGTTCT TGATACTGTA GAAAAATGGC |
| 8151 | TGGCTTGTGG GGTTGACAAC TTCTGTGTGA AGGTGTTAGC TCCATACATG |
| 8201 | CCAGATGTTC TCGAGAAACT GGAATTGCTC CAAAGGAGGT TTGGCGGAAC |
| 8251 | AGTGATCAGG AACCCTCTCT CCAGGAATTC CACTCATGAA ATGTACTACG |
| 8301 | TGTCTGGAGC CCGCAGCAAT GTCACATTTA CTGTGAACCA AACATCCCGC |
| 8401 | TGACGTCATC CTCCCAATTG GGACACGCAG TGTTGAGACA GACAAGGGAC |
| 8451 | CCCTGGACAA AGAGGCCATA GAAGAAAGGG TTGAGAGGAT AAAATCTGAG |
| 8501 | TACATGACCT CTTGGTTTTA TGACAATGAC AACCCCTACA GGACCTGGCA |
| 8551 | CTACTGTGGC TCCTATGTCA CAAAAACCTC AGGAAGTGCG GCGAGCATGG |
| 8601 | TAAATGGTGT TATTAAAATT CTGACATATC CATGGGACAG GATAGAGGAG |
| 8651 | GTCACCAGAA TGGCAATGAC TGACACAACC CCTTTTGGAC AGCAAAGAGT |
| 8701 | GTTTAAAGAA AAAGTTGACA CCAGAGCAAA GGATCCACCA GCGGGAACTA |
| 8751 | GGAAGATCAT GAAAGTTGTC AACAGGTGGC TGTTCCGCCA CCTGGCCAGA |
| 8801 | GAAAAGAGCC CCAGACTGTG CACAAAGGAA GAATTTATTG CAAAAGTCCG |
| 8851 | AAGTCATGCA GCCATTGGAG CTTACCTGGA AGAACAAGAA CAGTGGAAGA |
| 8901 | CTGCCAATGA GGCTGTCCAA GACCCAAAGT TCTGGGAACT GGTGGATGAA |
| 8951 | GAAAGGAAGC TGCACCAACA AGGCAGGTGT CGGACTTGTG TGTACAACAT |
| 9001 | GATGGGGAAA AGAGAGAAGA AGCTGTCAGA GTTTGGGAAA GCAAAGGGAA |
| 9051 | GCCGTGCCAT ATGGTATATG TGGCTGGGAG CGCGGTATCT TGAGTTTGAG |
| 9101 | GCCCTGGGAT TCCTGAATGA GGACCATTGG GCTTCCAGGG AAAACTCAGG |
| 9151 | AGGAGGAGTG GAAGGCATTG GCTTACAATA CCTAGGATAT GTGATCAGAG |
| 9201 | ACCTGGCTGC AATGGATGGT GGTGGATTCT ACGCGGATGA CACCGCTGGA |
| 9251 | TGGGACACGC GCATCACAGA GGCAGACCTT GATGATGAAC AGGAGATCTT |
| 9301 | GAACTACATG AGCCCACATC ACAAAAAACT GGCACAAGCA GTGATGGAAA |
| 9351 | TGACATACAA GAACAAAGTG GTGAAAGTGT TGAGACCAGC CCCAGGAGGG |
| 9401 | AAAGCCTACA TGGATGTCAT AAGTCGACGA GACCAGAGAG GATCCGGGCA |
| 9451 | GGTAGTGACT TATGCTCTGA ACACCATCAC CAACTTGAAA GTCCAATTGA |
| 9501 | TCAGAATGGC AGAAGCAGAG ATGGTGATAC ATCACCAACA TGTTCAAGAT |
| 9551 | TGTGATGAAT CAGTTCTGAC CAGGCTGGAG GCATGGCTCA CTGAGCACGG |
| 9601 | ATGTAACAGA CTGAAGAGGA TGGCGGTGAG TGGAGACGAC TGTGTGGTCC |
| 9651 | GGCCCATCGA TGACAGGTTC GGCCTGGCCC TGTCCCATCT CAACGCCATG |
| 9701 | TCCAAGGTTA GAAAGGACAT ATCTGAATGG CAGCCATCAA AAGGGTGGAA |
| 9751 | TGATTGGGAG AATGTGCCCT TCTGTTCCCA CCACTTCCAT GAACTACAGC |
| 9801 | TGAAGGATGG CAGGAGGATT GTGGTGCCTT GCCGAGAACA GGACGAGCTC |
| 9851 | ATTGGGAGAG GAAGGGTGTC TCCAGGAAAC GGCTGGATGA TCAAGGAAAC |
| 9901 | AGCTTGCCTC AGCAAAGCCT ATGCCAACAT GTGGTCACTG ATGTATTTTC |
| 9951 | ACAAAAGGGA CATGAGGCTA CTGTCATTGG CTGTTTCCTC AGCTGTTCCC |
| 10001 | ACCTCATGGG TTCCACAAGG ACGCACAACA TGGTCGATTC ATGGGAAAGG |
| 10051 | GGAGTGGATG ACCACGGAAG ACATGCTTGA GGTGTGGAAC AGAGTATGGA |
| 10101 | TAACCAACAA CCCACACATG CAGGACAAGA CAATGGTGAA AAAATGGAGA |
| 10151 | GATGTCCCTT ATCTAACCAA GAGACAAGAC AAGCTGTGCG GATCACTGAT |
| 10201 | TGGAATGACC AATAGGGCCA CCTGGGCCTC CCACATCCAT TTAGTCATCC |
| 10251 | ATCGTATCCG AACGCTGATT GGACAGGAGA AATACACTGA CTACCTAACA |
| 10301 | GTCATGGACA GGTATTCTGT GGATGCTGAC CTGCAACTGG GTGAGCTTAT |
| 10351 | CTGAAACACC ATCTAACAGG AATAACCGGG ATACAAACCA CGGGTGGAGA |
| 10401 | ACCGGACTCC CCACAACCTG AAACCGGGAT ATAAACCACG GCTGGAGAAC |
| 10451 | CGGACTCCGC ACTTAAAATG AAACAGAAAC CGGGATAAAA ACTACGGATG |
| 10501 | GAGAACCGGA CTCCACACAT TGAGACAGAA GAAGTTGTCA GCCCAGAACC |
| 10551 | CCACACGAGT TTTGCCACTG CTAAGCTGTG AGGCAGTGCA GGCTGGGACA |
| 10601 | GCCGACCTCC AGGTTGCGAA AAACCTGGTT TCTGGGACCT CCCACCCCAG |
| 10651 | AGTAAAAAGA ACGGAGCCTC CGCTACCACC CTCCCACGTG GTGGTAGAAA |
| 10701 | GACGGGGTCT AGAGGTTAGA GAAGACCCTC CAGGGAACAA ATAGTGGGAC |
| 10751 | CATATTGACG CCAGGGAAAG ACCGGAGTGG TTCTCTGCTT TTCCTCCAGA |
| 10801 | GGTCTGTGAG CACAGTTTGC TCAAGAATAA GCAGACCTTT GGATGACAAA |
| 10851 | CACAAAACCA CT |

1. A method for the production of Flavivirus as a vector for heterologous antigens comprising the introduction and expression of foreign gene sequences into insertion sites at the level of the envelope protein of any Flavivirus, wherein the sites are structurally apart from areas known to interfere with the overall flavivirus E protein structure and comprising:
(i) sites that lie on the external surface of the virus providing accessibility to antibody;
(ii) not disrupt or significantly destabilize the three-dimensional structure of the E protein; and,
(iii) not interfere with the formation of the E protein network within the viral envelope.

2. The method according to claim 1 wherein one site comprises the region of β-strands f and g including the fg loop which form part of the five-stranded anti-parallel β-sheet of domain II of the flavivirus envelope protein.

3. The method according to claim 2 wherein the site is the loop area between β-strands f and g which form part of the five-stranded anti-parallel β-sheet of domain II of the flavivirus envelope protein.

4. The method according to claim 2 wherein the foreign sequence has been inserted in the region of amino acid 196 to 215 with reference to the tick-borne encephalitis virus sequence described in FIG. 2.

5. The method according to claim 3 wherein the foreign sequence has been inserted in the region of amino acid 205 to 210 with reference to the tick-borne encephalitis virus sequence described in FIG. 2.

6. The method according to claim 1 wherein another site comprises the region of E₀ and F₀ strands including the E₀F₀ loop which form part of the eight stranded β-barrel of domain I.

7. The method according to claim 6 wherein the site is the loop area between E₀ and F₀ strands which form part of the eight stranded β-barrel of domain I.

8. The method according to claim 6 wherein the foreign sequence has been inserted in the region of amino acid 138 to 166 with reference to the tick-borne encephalitis virus sequence described in FIG. 2

9. The method according to claim 7 wherein the foreign sequence has been inserted in the region of amino acid 146 to 160 with reference to the tick-borne encephalitis virus sequence described in FIG. 2.

10. The method according to claim 1 wherein the Flavivirus is selected from the group consisting of any Flavivirus including yellow fever virus, tick borne encephalitis virus, dengue virus and japanese encephalitis virus.

11. The method according to claim 10 wherein the virus is a wild type, attenuated or recombinant virus.

12. The method according to claims 10 and 11 wherein the Flavivirus is a yellow fever virus.

13. The method according to claim 12 wherein the virus is a recombinant yellow fever virus.

14. The method according to claim 13 wherein the yellow fever virus is the YF17D virus strain and substrains thereof.

15. The method according to claims 1 to 14 wherein the foreign epitope is a malarial gene sequence.

16. The method according to claim 15 wherein the malarial gene sequence is the (NANP)₃ humoral epitope.

17. The method according to claim 15 wherein the malarial gene sequence is the DYENDIEKKI cytotoxic T-lymphocytes (CTL) epitope.

18. The method according to claim 15 wherein the malarial gene sequence is the SYVPSAEQI cytotoxic T-lymphocytes (CTL) epitope.

19. The method according to claim 1 wherein one or more glycine residues is inserted in the region immediately upstream and downstream of the foreign epitope.

20. A DNA construct consisting essentially of a vector, a genetically stable Flavivirus genome and foreign gene sequences introduced in an insertion site according to any of claims 1 to 19.

21. The DNA construct according to claim 20 wherein the Flavivirus is selected from the group consisting of any Flavivirus including yellow fever virus, tick borne encephalitis virus, dengue virus and japanese encephalitis virus

22. The DNA construct according to claim 20 wherein the vector is selected from the group consisting of low copy number plasmids.

23. The DNA construct according to claim 21 wherein the vector is selected from the group consisting of pACNR1180 and pBeloBAC11.

24. The DNA construct according to claim 20 wherein the vector is selected from the group consisting of high copy number plasmids.

25. The DNA construct according to claim 20 wherein the genetically stable Flavivirus genome is derived from any YF 17D strain.

26. The DNA construct according to claim 25 wherein the genetically stable Flavivirus genome is the YF genome bearing the complete sequence set forth in SEQ ID NO:1 or functionally equivalent sequences thereof.

27. The DNA construct according to claim 20 wherein the foreign gene sequence is derived from malaria, yellow fever, dengue, Japanese encephalitis, tick-borne encephalitis and fungi infections.

28. The DNA construct according to claim 27 wherein the foreign gene sequence is a malarial gene sequence.

29. The DNA construct according to claims 26 and 28 wherein the malarial gene sequence is the (NANP)₃ humoral epitope.

30. The DNA construct according to claim 28 wherein the malarial gene sequence is the DYENDIEKKI cytotoxic T-lymphocytes epitope.

31. The DNA construct according to claim 28 wherein the malarial gene sequence is the SYVPSAEQI cytotoxic T-lymphocytes epitope.

32. The DNA construct according to claim 29 which is plasmid pYF17D/8.

33. DNA construct having the structure of plasmid pYFE200.

34. DNA construct having the structure of plasmid pYFE200/1.

35. DNA construct having the structure of plasmid pYFE200/13.

36. DNA construct having the structure of plasmid pYFE200/8.

37. A Flavivirus as a vector for heterologous antigens comprising foreign gene sequences inserted at sites in the level of its envelope protein, wherein the sites are structurally apart from areas known to interfere with the overall flavivirus E protein structure.

38. The Flavivirus according to claim 37 wherein the foreign gene sequence is introduced in the region of β-strands f and g including the fg loop which form part of the five-stranded anti-parallel β-sheet of domain II of the flavivirus envelope protein.

39. The Flavivirus according to claim 38 wherein the site is the loop area between β-strands f and g which form part of the five-stranded anti-parallel β-sheet of domain II of the flavivirus envelope protein.

40. The Flavivirus according to claim 38 wherein the foreign sequence has been inserted in the region of amino acid 196 to 215 with reference to the tick-borne encephalitis virus sequence described in FIG. 2.

41. The Flavivirus according to claim 39 wherein the foreign sequence has been inserted in the region of amino acid 205 to 210 with reference to the tick-borne encephalitis virus sequence described in FIG. 2.

42. The Flavivirus according to claim 37 wherein another site comprises the region of E₀ and F₀ strands including the E₀F₀ loop which form part of the eight stranded β-barrel of domain I.

43. The Flavivirus according to claim 42 wherein the site is the loop area between E₀ and F₀ strands which form part of the eight stranded β-barrel of domain I.

44. The Flavivirus according to claim 42wherein the foreign sequence has been inserted in the region of amino acid 138 to 166 with reference to the tick-borne encephalitis virus sequence described in FIG. 2.

45. The Flavivirus according to claim 43 wherein the foreign sequence has been inserted in the region of amino acid 146 to 160 with reference to the tick-borne encephalitis virus sequence described in FIG. 2.

46. The Flavivirus according to claim 37 wherein the Flavivirus is selected from the group consisting of any Flavivirus including yellow fever virus, tick borne encephalitis virus, dengue virus and japanese encephalitis virus.

47. The Flavivirus according to claim 46 wherein the virus is a wild type, attenuated or recombinant virus.

48. The Flavivirus according to claims 46 and 47 wherein the Flavivirus is a yellow fever virus.

49. The Flavivirus according to claim 48 wherein the virus is a recombinant yellow fever virus.

50. The Flavivirus according to claim 49 wherein the yellow fever virus is the YF17D virus strain and substrains thereof.

51. The Flavivirus according to claims 37 to 50 wherein the foreign epitope is a malarial gene sequence.

52. A vaccine composition to immunize against flavivirus and other infectious agents consisting essentially of a virus according to claims 37 to 51.

53. The vaccine composition according to claim 52 wherein the flavivirus is a yellow fever virus and the other infectious agent is the causative agent of malaria.

54. The vaccine composition according to claim 53 wherein the malarial gene sequence is the (NANP)₃ humoral epitope.

55. The vaccine composition according to claim 53 wherein the malarial gene sequence is the DYENDIEKKI cytotoxic T-lymphocytes epitope.

56. The vaccine composition according to claim 53 wherein the malarial gene sequence is the SYVPSAEQI cytotoxic T-lymphocytes epitope.

57. The vaccine composition according to claims 37 to 51 comprising a sufficient amount of the virus and a pharmaceutically acceptable vehicle.

58. A Flavivirus as a vector for heterologous antigens wherein the Flavivirus is obtainable according to any of claims 1 to 19.
